SEQUENCE LISTING (38 sequences)

SEQ ID NO: 1 - protein, 554 aa, Homo sapiens
  Human soluble epoxide hydrolase (sEH)
MTLRGAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGWPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 2 - DNA, 1665 bp, Homo sapiens
  Human soluble epoxide hydrolase (sEH)
atgacgctgcgcggcgccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctggcctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtag

SEQ ID NO: 3 - DNA, 23 bp, Artificial Sequence
  Description of Artificial Sequence Synthetic soluble epoxide hydrolase (sEH) target sequence
cagtgttcattggccatgactgg

SEQ ID NO: 4 - DNA, 21 bp, Artificial Sequence
  Description of Artificial Sequence Synthetic soluble epoxide hydrolase (sEH) sense-small interfering RNA (siRNA)
guguucauuggccaugacutt

SEQ ID NO: 5 - DNA, 21 bp, Artificial Sequence
  Description of Artificial Sequence Synthetic soluble epoxide hydrolase (sEH) antisense-small interfering RNA (siRNA)
agucauggccaaugaacactt

SEQ ID NO: 6 - DNA, 23 bp, Artificial Sequence
  Description of Artificial Sequence Synthetic soluble epoxide hydrolase (sEH) target sequence
gaaaggctatggagagtcatctg

SEQ ID NO: 7 - DNA, 21 bp, Artificial Sequence
  Description of Artificial Sequence Synthetic soluble epoxide hydrolase (sEH) sense-small interfering RNA (siRNA)
aaggcuauggagagucauctt

SEQ ID NO: 8 - DNA, 21 bp, Artificial Sequence
  Description of Artificial Sequence Synthetic soluble epoxide hydrolase (sEH) antisense-small interfering RNA (siRNA)
gaugacucuccauagccuutt

SEQ ID NO: 9 - DNA, 23 bp, Artificial Sequence
  Description of Artificial Sequence Synthetic soluble epoxide hydrolase (sEH) target sequence
aaaggctatggagagtcatctgc

SEQ ID NO: 10 - DNA, 21 bp, Artificial Sequence
  Description of Artificial Sequence Synthetic soluble epoxide hydrolase (sEH) sense-small interfering RNA (siRNA)
aggcuauggagagucaucutt

SEQ ID NO: 11 - DNA, 21 bp, Artificial Sequence
  Description of Artificial Sequence Synthetic soluble epoxide hydrolase (sEH) antisense-small interfering RNA (siRNA)
agaugacucuccauagccutt

SEQ ID NO: 12 - DNA, 23 bp, Artificial Sequence
  Description of Artificial Sequence Synthetic soluble epoxide hydrolase (sEH) target sequence
caagcagtgttcattggccatga

SEQ ID NO: 13 - DNA, 21 bp, Artificial Sequence
  Description of Artificial Sequence Synthetic soluble epoxide hydrolase (sEH) sense-small interfering RNA (siRNA)
agcaguguucauuggccautt

SEQ ID NO: 14 - DNA, 21 bp, Artificial Sequence
  Description of Artificial Sequence Synthetic soluble epoxide hydrolase (sEH) antisense-small interfering RNA (siRNA)
auggccaaugaacacugcutt

SEQ ID NO: 15 - DNA, 23 bp, Artificial Sequence
  Description of Artificial Sequence Synthetic soluble epoxide hydrolase (sEH) target sequence
cagcacatggaggactggattcc

SEQ ID NO: 16 - DNA, 21 bp, Artificial Sequence
  Description of Artificial Sequence Synthetic soluble epoxide hydrolase (sEH) sense-small interfering RNA (siRNA)
gcacauggaggacuggauutt

SEQ ID NO: 17 - DNA, 21 bp, Artificial Sequence
  Description of Artificial Sequence Synthetic soluble epoxide hydrolase (sEH) antisense-small interfering RNA (siRNA)
aauccaguccuccaugugctt

SEQ ID NO: 18 - DNA, 9 bp, Artificial Sequence
  Description of Artificial Sequence Synthetic short spacer linking sense siRNA to reverse complementary antisense siRNA
ttcaagaga

SEQ ID NO: 19 - DNA, 23 bp, Artificial Sequence
  Description of Artificial Sequence Synthetic soluble epoxide hydrolase (sEH) short hairpin small interfering RNA (siRNA) target sequence
cagtgttcattggccatgactgg

SEQ ID NO: 20 - DNA, 59 bp, Artificial Sequence
  Description of Artificial Sequence Synthetic soluble epoxide hydrolase (sEH) short hairpin small interfering RNA (siRNA) sense strand
gatccccgtgttcattggccatgactttcaagagaagtcatggccaatgaacacttttt

SEQ ID NO: 21 - DNA, 59 bp, Artificial Sequence
  Description of Artificial Sequence Synthetic soluble epoxide hydrolase (sEH) short hairpin small interfering RNA (siRNA) antisense strand
agctaaaaagtgttcattggccatgacttctcttgaaagtcatggccaatgaacacggg

SEQ ID NO: 22 - DNA, 23 bp, Artificial Sequence
  Description of Artificial Sequence Synthetic soluble epoxide hydrolase (sEH) short hairpin small interfering RNA (siRNA) target sequence
gaaaggctatggagagtcatctg

SEQ ID NO: 23 - DNA, 59 bp, Artificial Sequence
  Description of Artificial Sequence Synthetic soluble epoxide hydrolase (sEH) short hairpin small interfering  RNA (siRNA) sense strand
gatccccaaggctatggagagtcatcttcaagagagatgactctccatagccttttttt

SEQ ID NO: 24 - DNA, 59 bp, Artificial Sequence
  Description of Artificial Sequence Synthetic soluble epoxide hydrolase (sEH) short hairpin small interfering  RNA (siRNA) antisense strand
agctaaaaaaaggctatggagagtcatctctcttgaagatgactctccatagccttggg

SEQ ID NO: 25 - DNA, 23 bp, Artificial Sequence
  Description of Artificial Sequence Synthetic soluble epoxide hydrolase (sEH) short hairpin small interfering  RNA (siRNA) target sequence
aaaggctatggagagtcatctgc

SEQ ID NO: 26 - DNA, 59 bp, Artificial Sequence
  Description of Artificial Sequence Synthetic soluble epoxide hydrolase (sEH) short hairpin small interfering  RNA (siRNA) sense strand
gatccccaggctatggagagtcatctttcaagagaagatgactctccatagcctttttt

SEQ ID NO: 27 - DNA, 59 bp, Artificial Sequence
  Description of Artificial Sequence Synthetic soluble epoxide hydrolase (sEH) short hairpin small interfering  RNA (siRNA) antisense strand
agctaaaaaaggctatggagagtcatcatctcttgaaagatgactctccatagcctggg

SEQ ID NO: 28 - DNA, 23 bp, Artificial Sequence
  Description of Artificial Sequence Synthetic soluble epoxide hydrolase (sEH) short hairpin small interfering  RNA (siRNA) target sequence
caagcagtgttcattggccatga

SEQ ID NO: 29 - DNA, 59 bp, Artificial Sequence
  Description of Artificial Sequence Synthetic soluble epoxide hydrolase (sEH) short hairpin small interfering  RNA (siRNA) sense strand
gatccccagcagtgttcattggccatttcaagagaatggccaatgaacactgctttttt

SEQ ID NO: 30 - DNA, 59 bp, Artificial Sequence
  Description of Artificial Sequence Synthetic soluble epoxide hydrolase (sEH) short hairpin small interfering  RNA (siRNA) antisense strand
agctaaaaaagcagtgttcattggccattctcttgaaatggccaatgaacactgctggg

SEQ ID NO: 31 - DNA, 23 bp, Artificial Sequence
  Description of Artificial Sequence Synthetic soluble epoxide hydrolase (sEH) short hairpin small interfering  RNA (siRNA) target sequence
cagcacatggaggactggattcc

SEQ ID NO: 32 - DNA, 59 bp, Artificial Sequence
  Description of Artificial Sequence Synthetic soluble epoxide hydrolase (sEH) short hairpin small interfering  RNA (siRNA) sense strand
gatccccgcacatggaggactggattttcaagagaaatccagtcctccatgtgcttttt

SEQ ID NO: 33 - DNA, 59 bp, Artificial Sequence
  Description of Artificial Sequence Synthetic soluble epoxide hydrolase (sEH) short hairpin small interfering  RNA (siRNA) antisense strand
agctaaaaagcacatggaggactggatttctcttgaaaatccagtcctccatgtgcggg

SEQ ID NO: 34 - RNA, 20 nt, Artificial Sequence
  Description of Artificial Sequence Synthetic soluble epoxide hydrolase (sEH) antisense sequence
uguccagugcccacaguccu

SEQ ID NO: 35 - RNA, 20 nt, Artificial Sequence
  Description of Artificial Sequence Synthetic soluble epoxide hydrolase (sEH) antisense sequence
uucccaccugacacgacucu

SEQ ID NO: 36 - RNA, 20 nt, Artificial Sequence
  Description of Artificial Sequence Synthetic soluble epoxide hydrolase (sEH) antisense sequence
guucagccucagccacuccu

SEQ ID NO: 37 - RNA, 19 nt, Artificial Sequence
  Description of Artificial Sequence Synthetic soluble epoxide hydrolase (sEH) antisense sequence
aguccucccgcuucacaga

SEQ ID NO: 38 - RNA, 21 nt, Artificial Sequence
  Description of Artificial Sequence Synthetic soluble epoxide hydrolase (sEH) antisense sequence
gcccacuuccaguuccuuucc

(19) United States
(12) Patent Application Publication
(10) Pub. No.: US 2011/0230504 A1
(43) Pub. Date: Sep. 22, 2011

(54) ALLEVIATING DISORDERS WITH COMBINING AGENTS THAT INCREASE EPOXYGENATED FATTY ACIDS AND AGENTS THAT INCREASE cAMP

(75) Inventors: Bruce D. Hammock, Davis, CA (US); Ahmet Bora Inceoglu, Davis, CA (US); Steven L. Jinks, Davis, CA (US)

(73) Assignee: The Regents Of The University Of California Office Of Technology, Oakland, CA (US)

(21) Appl. No.: 13/063,459

(22) PCT Filed: Sep. 11, 2009
(86) PCT No.: PCT/US09/56617

§ 371 Date: May 12, 2011

Related U.S. Application Data
(60) Provisional application No. 61/097,141, filed on Sep. 15, 2008.

(51) Int. Cl. A61K 31/522; A61K 31/336; A61K 31/4015; A61K 31/44; A61K 31/277; A61K 31/451; A61K 31/437; A61K 31/403; A61P 29/00; A61P 23/00; A61P 25/24; A61P 25/08

(52) U.S. Cl. 514/263.36; 514/475; 514/424; 514/352; 514/567; 514/327; 514/300; 514/412

(57) ABSTRACT

The present invention relates to compositions and methods for promoting and enhancing the analgesic, anesthetic and anticonvulsant properties of epoxygenated fatty acids, in particular, epoxy-eicosatrienoic acids (“EETs”) and inhibitors of soluble epoxide hydrolase (“sEH”) in the presence of elevated levels of cyclic adenosine monophosphate (“cAMP”) by combining or co-administering the epoxygenated fatty acid, EET and/or inhibitor of sEH with an agent that increases intracellular levels of cAMP, e.g., a phosphodiesterase inhibitor.

### CROSS-REFERENCES TO RELATED APPLICATIONS

This application claims the benefit of U.S. Provisional Application No. 61/097,141, filed on Sep. 15, 2008, the entire disclosure of which is hereby incorporated herein by reference.

### STATEMENT AS TO RIGHTS TO INVENTIONS MADE UNDER FEDERALLY SPONSORED RESEARCH AND DEVELOPMENT

This invention was made with government support with National Institute on Environmental Health Sciences Grant R37 ES02710, National Institute on Environmental Health Sciences Superfund Basic Research Program Grant P42 ES04699, National Institutes of Health Grant HL 59699, and National Institutes of Health Grant GM 78167. The government has certain rights in the invention.

### FIELD OF THE INVENTION

The present invention relates to compositions and methods for promoting and enhancing the analgesic, anesthetic and anticonvulsant properties of epoxygenated fatty acids, in particular, epoxy-eicosatrienoic acids (“EETs”) and inhibitors of soluble epoxide hydrolase (“sEH”) in the presence of elevated levels of cyclic adenosine monophosphate (“cAMP”) by combining or co-administering the epoxygenated fatty acid, EET and/or inhibitor of sEH with an agent that increases intracellular levels of cAMP, e.g., a phosphodiesterase inhibitor.

### BACKGROUND OF THE INVENTION

Inflammation and pain are debilitating factors associated with a multitude of diseases. Although many therapeutic agents for control of pain are available, side effects and lack of wide spectrum efficacy call for a better understanding of biological events governing diverse classes of facilitated pain states. The arachidonic acid (AA) cascade for example is a relatively well inflammation and pain. Being a substrate for cyclooxygenases (cox), lipoxygenases and cytochrome P450 family enzymes released AA is converted to an expanding number of known lipid mediators including prostaglandins, leukotrienes and EETs (Vane J R et al., Annu Rev Pharmacol Toxicol 38:97-120 (1998); Capdevila J H et al., FASEB J 6:731-736 (1992)). While some of these mediators drive inflammation others limit or resolve it (Serhan C N et al., Nat Rev Immunol 8:349-361 (2008)). Inflammatory pain is well correlated with the production of prostaglandins, cox-2 metabolites of AA both in the central nervous system and the periphery (Ferreira S H et al., Eur J Pharmacol 53:39-48 (1978)). As a result inhibition of the inducible cox-2 leads to relief from inflammatory pain which is often attributed to the decreased production of prostaglandin E₂ (PGE₂) (Vane J R, Nat New Biol 231:232-235 (1971)). The lesser appreciated branch of the AA cascade is the cytochrome P450 pathway in which the known major endogenous products are 20-HETE, a potent hypertensive and proinflammatory mediator, and EETs (McGiff J C, Annu Rev Pharmacol Toxicol 31:339-369 (1991); Spector A A and Norris A W, Am J Physiol Cell Physiol 292:C996-C1012 (2006); Campbell W et al., Endocrinology 128:2183-2194 (1991)). The EETs are widely assumed to be a major component of the vascular endothelium derived hyperpolarizing factor and have further effects including ion channel modulation and regulation of gene expression (Spector A A and Norris A W, Am J Physiol Cell Physiol 292:C996-C1012 (2006); Fisslthaler B et al., Nature 401:493-497 (1999); Campbell W et al., Circ Res 78:415-423 (1996); Node K et al., Science 285:1276-1279 (1999)). Strong anti-inflammatory activity of EETs is indicated through their ability to inhibit nuclear translocation of NF-κB (Node K et al., Science 285:1276-1279 (1999)). Recently EETs have been demonstrated to be antinociceptive when administered directly into the brain as well (Terashvili M et al., J Pharmacol Exp Ther 326:614-622 (2008)). The predicted in vivo half-lives of EETs are in the order of seconds, largely due to rapid conversion to the corresponding diols or DHETs (dihydroeicosatrienoic acids) by the soluble epoxide hydrolase (sEH). However EETs are stabilized using inhibitors of sEH (sEHI) that prevent the conversion of EETs to corresponding diols (Spector A A and Norris A W, Am J Physiol Cell Physiol 292:C996-C1012 (2006)). The increased EETs then lead to a reduction in blood pressure during hypertension and to antihyperalgesia during inflammation whereas the diols are thought to be less active (Spector A A and Norris A W, Am J Physiol Cell Physiol 292:C996-C1012 (2006); Inceoglu B et al., Life Sci 79:2311-2319 (2006); Inceoglu B et al., Prostag Other Lipid Mediat 82:42-49 (2007)). Although many in vitro biological activities of EETs are characterized, the ability to inhibit sEH in vivo provides the advantage of revealing the systemic physiological effects of these molecules. Here we present evidence towards two distinct mechanisms by which EETs modulate nociceptive pathways by altering transcriptional plasticity in the spinal cord and the brain.

The enzyme soluble epoxide hydrolase is thought to have a key role in regulating a group of bioactive lipid metabolites, the epoxygenated fatty acids, by effectively degrading these potent biomolecules to inactive or less active metabolites (Spector A A and Norris A W, American Journal of Physiology-Cell Physiology 00402.02006 (2006)). Consistent with the diversity of epoxygenated fatty acids and their potential biological roles, in vivo inhibition of sEH results in a wide variety of biological outcomes in distinct disease models (Inceoglu B et al., Prostaglandins & Other Lipid Mediators 82:42-49 (2007)). For example, in multiple rodent models, inhibitors of sEH are anti-hypertensive, cardio-protective in heart disease models and antihyperalgesic during inflammatory pain (Imig J D et al., Hypertension 39:690-694 (2002); Inceoglu B et al., Life Sciences 79:2311-2319 (2006); Schmelzer K R et al., Proc Natl Acad Sci USA September 12; ( ): 103:13646-13651 (2006)). However, inhibitors of sEH (sEHi) do not seem to have an effect on blood pressure in normotensive animals, nor do they change nociceptive thresholds of rats in the absence of persistent pain states. The diverse biological activities of inhibiting sEH, in most cases, is linked to increases in the levels of the epoxygenated arachidonic acid metabolites, epoxyeicosatrienoic acids (EETs), which are among the endogenous substrates of the sEH. Remarkably, in parallel to the predicted activities of natural EETs, chemical inhibition of sEH by synthetic inhibitors or genetic inhibition by knocking down the sEH gene effectively increases the levels of EETs and results in anti-inflammatory effects and alleviates inflammatory hyperalgesia (Inceoglu B et al., Life Sciences 79:2311-2319 (2006)).

Although numerous in vitro effects of EETs are known few in vivo effects are characterized. This is mainly because EETs have half lives in the order of seconds unless stabilized by sEHi. Recently developed potent and bioavailable inhibitors of sEH enabled the demonstration of several novel biological effects of increasing EET levels (Jones P D et al., Bioorganic & Medicinal Chemistry Letters 16:5212-5216 (2006); Inceoglu B et al., Prostaglandins & Other Lipid Mediators 82:42-49 (2007)). Most prominently, anti-inflammatory and antinociceptive effects for both EETs and sEHi in rodent models of systemic and local inflammation were demonstrated (Inceoglu B et al., Life Sciences 79:2311-2319 (2006); Schmelzer K R et al., Proc Natl Acad Sci USA September 12; ( ): 103:13646-13651 (2006)). These effects were intriguing because the sEH, a soluble and largely cytosolic enzyme, though expressed selectively in the CNS, is not a protein that was previously associated with sensory function (Sura P et al., J Histochem Cytochem 56:551-559 (2008)). However, structurally different sEHi that penetrate into the CNS strongly reduce hyperalgesia and suppress the induction of COX2 gene in the spinal cord of inflamed rats (Inceoglu B et al., Proc Natl Acad Sci USA 105:18901-18906 (2008)). In inflamed animals treated with sEHi, consistent with the suppression of COX2, plasma levels of prostaglandins are also reduced (Inceoglu B et al., Life Sciences 79:2311-2319 (2006)). Although it is not known how sEHi or EETs suppress COX2 transcription, EETs have been shown to prevent NF-κB translocation and therefore the suppression could be dependent on this activity (Node K et al., Science 285:1276-1279 (1999)).

Interestingly, recent studies suggest a direct role for EETs in nociceptive signaling. Specifically an EET regioisomer, 14,15-EET, has been shown to produce antinociception by activating endorphin release when administered into the ventrolateral periaqueductal gray of the brain (Terashvili M et al., J Pharmacol Exp Ther 326:614-622 (2008)). Several sEHi effectively penetrate into the CNS (Inceoglu B et al., Proc Natl Acad Sci USA 105:18901-18906 (2008)). However, the lack of sEHi effect in non-inflamed animals and the antihyperalgesic effects during inflammatory pain is still unexplained. We hypothesized the requirement of a factor(s) that is brought about by the disease state and released into the cellular environment for the EETs to act upon or to act together with. Because arachidonic acid is the precursor to EETs and is released in large quantities in the course of inflammation, it is plausible that arachidonic acid is at least one of these factors.

However, we also found, in the CNS, elevated cAMP could be another requirement. In the CNS EETs or sEHi acted cooperatively with inflammation driven intracellular cAMP or with intraspinally administered db-cAMP to enhance brain and spinal cord StARD1 (steroidogenic acute regulatory protein) expression (Inceoglu B et al., Proc Natl Acad Sci USA 105:18901-18906 (2008)). The steroidogenic gene StARD1 is a required acute steroid producing gene for the first and the rate limiting step in steroidogenesis, the transport of cholesterol from the outer to the inner membrane of mitochondria (Clark B J et al., Characterization of the steroidogenic acute regulatory protein (StAR) 269:28314-28322 (1994); Bose H S et al., Nature 417:87-91 (2002); Papadopoulos V L et al., Neuroscience 138:749-756 (2006)). Steroidogenesis was initially thought to be stimulated by arachidonic acid, a key regulatory lipid (Lin T, Life Sciences 36:1255-1264 (1985)). However, later arachidonic acid metabolites, in particular EETs were implicated at least for part of this activity when they were found to increase steroid production and enhanced the in vitro expression of StARD1 in cultured mouse Leydig cells (Nishimura M et al., Prostaglandins 38:413-430 (1989); Van Voorhis B J et al., J Clin Endocrinol Metab 76:1555-1559 (1993); Wang X et al., The involvement of epoxygenase metabolites of arachidonic acid in cAMP-stimulated steroidogenesis and steroidogenic acute regulatory protein gene expression, 190:871-878 (2006)). Although a large body of literature exists on steroid synthesis in the steroidogenic tissues, acute steroidogenesis in the CNS is much less known; though it is thought to proceed in parallel to that in other steroidogenic cells (Do Rego J L et al., Frontiers in Neuroendocrinology In Press, Corrected Proof; Furukawa A et al., Steroidogenic Acute Regulatory Protein (StAR) Transcripts Constitutively Expressed in the Adult Rat Central Nervous System Colocalization of StAR, Cytochrome P-450SCC (CYP XIA1), and 3&#x03B2; -Hydroxysteroid Dehydrogenase in the Rat Brain 71:2231-2238 (1998)). In the CNS, similar steroidogenic molecular machinery is proposed to produce neuroactive steroids that are known to have analgesic, anti-convulsant, sedative, hypnotic, anesthetic and anxiolytic properties, primarily through their actions on GABA_{A} receptor conductance (King S R et al., J Neurosci 22:10613-10620 (2002); Sanna E et al., The Journal of Neuroscience 24:6521-6530 (2004); Belelli D and Lambert J J, Nature Reviews Neuroscience 6:565-575 (2005); Verleye M et al., Pharmacology Biochemistry and Behavior 82:712-720 (2005); Morrow A L, Pharmacology & Therapeutics 116:1-6 (2007)). Here, we tested the hypothesis that inhibition of sEH will lead to antinociception and whether these effects are associated with upregulating spinal and supraspinal StARD1 or neurosteroid production when intracellular cAMP levels are concomitantly increased by inhibiting PDE.

### BRIEF SUMMARY OF THE INVENTION

The present invention is based, in part, on the unexpected discovery that that the analgesic, anesthetic and anticonvulsant effects of epoxygenated fatty acids, in particular epoxy-eicosatrienoic acids (“EETs”), and inhibitors of soluble epoxide hydrolase (“sEH”) are enhanced in the presence of elevated levels of cyclic adenosine monophosphate (“cAMP”). Accordingly, in one aspect, the invention provides compositions. In some embodiments, the compositions comprise (a) (i) an inhibitor of soluble epoxide hydrolase (“sEH”), (ii) an epoxygenated fatty acid, or (iii) both an inhibitor of sEH and an epoxygenated fatty acid, and (b) an agent that increases intracellular levels of cyclic adenosine monophosphate (“cAMP”). In some embodiments, the agents in the compositions are provided as a mixture.

In a further aspect, the invention provides methods of reducing the severity and/or frequency of seizures in a subject in need thereof. In some embodiments, the methods comprise co-administering (a) (i) an inhibitor of sEH, (ii) an epoxygenated fatty acid, or (iii) both an inhibitor of sEH and an epoxygenated fatty acid, and (b) an agent that increases intracellular levels of cAMP. In some embodiments, the subject has a form of epilepsy. In some embodiments, the agents are co-administered with a neurosteroid. In some embodiments, the form of epilepsy is status epilepticus.

In a related aspect, the invention provides methods for reducing depression, seizures in subjects with epilepsy, or of providing post-surgical analgesia during recovery from anesthesia. In some embodiments, the methods comprise co-administering (a) (i) an inhibitor of sEH, (ii) an epoxygenated fatty acid, or (iii) both an inhibitor of sEH and an epoxygenated fatty acid, and (b) an agent that increases intracellular levels of cAMP. In some embodiments, the form of epilepsy is status epilepticus.

In another aspect, the invention provides methods of enhancing the analgesic effects of EETs and inhibitors of sEH in a subject in need thereof. In some embodiments, the methods comprise co-administering (a) (i) an inhibitor of sEH, (ii) an epoxygenated fatty acid, or (iii) both an inhibitor of sEH and an epoxygenated fatty acid, and (b) an agent that increases intracellular levels of cAMP. In some embodiments, the agents are co-administered with a neurosteroid.

In a further aspect, the invention provides methods of enhancing anesthesia in a subject in need thereof. In some embodiments, the methods comprise co-administering (a) (i) an inhibitor of sEH, (ii) an epoxygenated fatty acid, or (iii) both an inhibitor of sEH and an epoxygenated fatty acid, and (b) an agent that increases intracellular levels of cAMP. In some embodiments, the anesthesia is induced by a barbiturate. In some embodiments, the agents are co-administered with a barbiturate. In some embodiments, the agents are co-administered with a neurosteroid.

With respect to the embodiments, in some embodiments, the epoxygenated fatty acid is an epoxy-eicosatrienoic acid (“EET”). Exemplary EETs that find use include 14,15-EET, 8,9-EET and 11,12-EET, and 5,6 EETs.

In some embodiments, the epoxygenated fatty acid is an epoxide of linoleic acid, eicosapentaenoic acid (“EPA”) or docosahexaenoic acid (“DHA”), or a mixture thereof.

In some embodiments, the inhibitor of sEH has a primary pharmacophore selected from the group consisting of a urea, a carbamate, a piperidine and an amide.

In some embodiments, the agent that increases intracellular levels of cAMP is an inhibitor of phosphodiesterase. In some embodiments, the inhibitor of phosphodiesterase is a non-selective inhibitor of phosphodiesterase. In some embodiments, the PDE inhibitor specifically or preferentially inhibits a cAMP PDE, e.g., inhibits PDE4, PDE7 or PDE8. In some embodiments, the PDE inhibitor used inhibits a cAMP PDE, e.g., inhibits PDE1, PDE2, PDE3, PDE4, PDE7, PDE8, PDE10 or PDE11.

In some embodiments, the inhibitor of phosphodiesterase is an inhibitor of PDE4. Exemplary inhibitors of PDE4 that find use include without limitation, rolipram, roflumilast, cilomilast, ariflo, HT0712, ibudilast, mesembrine, pentoxifylline, piclamilast, and combinations thereof. In some embodiments, the inhibitor of phosphodiesterase is rolipram.

In some embodiments, the inhibitor of phosphodiesterase is an inhibitor of PDE5. Exemplary inhibitors of PDE5 that find use include without limitation, sildenafil, zaprinast, tadalafil, vardenafil and combinations thereof.

The agents can be concurrently or sequentially administered. The agents can be administered by the same or different route of administration.

In some embodiments, the subject or patient is a human.

### Definitions

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“cis-Epoxyeicosatrienoic acids” (“EETs”) are biomediators synthesized by cytochrome P450 epoxygenases. As discussed further in a separate section below, while the use of unmodified EETs is the most preferred, derivatives of EETs, such as amides and esters (both natural and synthetic), EETs analogs, and EETs optical isomers can all be used in the methods of the invention, both in pure form and as mixtures of these forms. For convenience of reference, the term “EETs” as used herein refers to all of these forms unless otherwise required by context.

“Epoxide hydrolases” (“EH;” EC 3.3.2.3) are enzymes in the alpha beta hydrolase fold family that add water to 3-membered cyclic ethers termed epoxides. The addition of water to the epoxides results in the corresponding 1,2-diols (Hammock, B. D. et al., in Comprehensive Toxicology: Biotransformation (Elsevier, New York), pp. 283-305 (1997); Oesch, F. Xenobiotica 3:305-340 (1972)). Four principal EH's are known: leukotriene epoxide hydrolase, cholesterol epoxide hydrolase, microsomal EH (“mEH”), and soluble EH (“sEH,” previously called cytosolic EH). The leukotriene EH acts on leukotriene A4, whereas the cholesterol EH hydrates compounds related to the 5,6-epoxide of cholesterol. The microsomal epoxide hydrolase metabolizes monosubstituted, 1,1-disubstituted, cis-1,2-disubstituted epoxides and epoxides on cyclic systems to their corresponding diols. Because of its broad substrate specificity, this enzyme is thought to play a significant role in ameliorating epoxide toxicity. Reactions of detoxification typically decrease the hydrophobicity of a compound, resulting in a more polar and thereby excretable substance.

“Soluble epoxide hydrolase” (“sEH”) is an epoxide hydrolase which in many cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). NCBI Entrez Nucleotide accession number L05779 sets forth the nucleic acid sequence encoding the protein, as well as the 5′ untranslated region and the 3′ untranslated region. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Soluble EH is only very distantly related to mEH and hydrates a wide range of epoxides not on cyclic systems. In contrast to the role played in the degradation of potential toxic epoxides by mEH, sEH is believed to play a role in the formation or degradation of endogenous chemical mediators. Unless otherwise specified, as used herein, the terms “soluble epoxide hydrolase” and “sEH” refer to human sEH.

Unless otherwise specified, as used herein, the terms “sEH inhibitor” (also abbreviated as “sEHI”) or “inhibitor of sEH” refer to an inhibitor of human sEH. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

The term “neuroactive steroid” or “neurosteroids” interchangeably refer to steroids that rapidly alter neuronal excitability through interaction with neurotransmitter-gated ion channels, and which may also exert effects on gene expression via intracellular steroid hormone receptors.

Neurosteroids have a wide range of applications from sedation to treatment of epilepsy and traumatic brain injury. Neurosteroids can act as allosteric modulators of neurotransmitter receptors, such as GABA_{A}, NMDA, and sigma receptors. Progesterone (PROG) is also a neurosteroid which activates progesterone receptors expressed in peripheral and central glial cells. Several synthetic neurosteroids have been used as sedatives for the purpose of general anaesthesia for carrying out surgical procedures. Exemplary sedating neurosteroids include without limitation alphaxolone, alphadolone, hydroxydione and minaxolone. The neurosteroid ganaxolone finds use for the treatment of epilepsy.

The term “epilepsy” refers to a chronic neurological disorder characterized by recurrent unprovoked seizures. These seizures are transient signs and/or symptoms of abnormal, excessive or synchronous neuronal activity in the brain. There are over 40 different types of epilepsy, including without limitation absence seizures, atonic seizures, benign Rolandic epilepsy, childhood absence, clonic seizures, complex partial seizures, frontal lobe epilepsy, Febrile seizures, Infantile spasms, Juvenile Myoclonic Epilepsy, Juvenile Absence Epilepsy, lennox-gastaut syndrom, Landau-Kleffner Syndrome, myoclonic seizures, Mitochondrial Disorders, Progressive Myoclonic Epilepsies, Psychogenic Seizures, Reflex Epilepsy, Rasmussen's Syndrome, Simple Partial seizures, Secondarily Generalized Seizures, Temporal Lobe Epilepsy, Toni-clonic seizures, Tonic seizures, Psychomotor Seizures, Limbic Epilepsy, Partial-Onset Seizures, generalised-onset seizures, Status Epilepticus, Abdominal Epilepsy, Akinetic Seizures, Auto-nomic seizures, Massive Bilateral Myoclonus, Catamenial Epilepsy, prop seizures, Emotional seizures, Focal seizures, Gelastic seizures, Jacksonian March, Lafora Disease, Motor seizures, Multifocal seizures, Neonatal seizures, Nocturnal seizures, Photosensitive seizure, Pseudo seizures, Sensory seizures, Subtle seizures, Sylvan Seizures, Withdrawal seizures and Visual Reflex Seizures. The most widespread classification of the epilepsies divides epilepsy syndromes by location or distribution of seizures (as revealed by the appearance of the seizures and by EEG) and by cause. Syndromes are divided into localization-related epilepsies, generalized epilepsies, or epilepsies of unknown localization. Localization-related epilepsies, sometimes termed partial or focal epilepsies, arise from an epileptic focus, a small portion of the brain that serves as the irritant driving the epileptic response. Generalized epilepsies, in contrast, arise from many independent foci (multifocal epilepsies) or from epileptic circuits that involve the whole brain. Epilepsies of unknown localization remain unclear whether they arise from a portion of the brain or from more widespread circuits. Epilepsy syndromes are further divided by presumptive cause: idiopathic, symptomatic, and cryptogenic. Idiopathic epilepsies are generally thought to arise from genetic abnormalities that lead to alteration of basic neuronal regulation. Symptomatic epilepsies arise from the effects of an epileptic lesion, whether that lesion is focal, such as a tumor, or a defect in metabolism causing widespread injury to the brain. Cryptogenic epilepsies involve a presumptive lesion that is otherwise difficult or impossible to uncover during evaluation. Forms of epilepsy are well characterized and review, e.g., in Epilepsy: A Comprehensive Textbook (3-volume set), Engel, et al., editors, 2^{nd} Edition, 2007, Lippincott, Williams and Wilkins; and The Treatment of Epilepsy: Principles and Practice, Wyllie, et al., editors, 4^{th} Edition, 2005, Lippincott, Williams and Wilkins; and Browne and Holmes, Handbook of Epilepsy, 4^{th} Edition, 2008, Lippincott, Williams and Wilkins.

By “physiological conditions” is meant an extracellular milieu having conditions (e.g., temperature, pH, and osmolarity) which allows for the sustenance or growth of a cell of interest.

“Micro-RNA” (“miRNA”) refers to small, noncoding RNAs of 18-25 nt in length that negatively regulate their complementary mRNAs at the posttranscriptional level in many eukaryotic organisms. See, e.g., Kurihara and Watanabe, Proc Natl Acad Sci USA 101(34):12753-12758 (2004). Micro-RNA's were first discovered in the roundworm C. elegans in the early 1990s and are now known in many species, including humans. As used herein, it refers to exogenously administered miRNA unless specifically noted or otherwise required by context.

The term “co-administration” refers to the presence of both active agents in the blood at the same time. Active agents that are co-administered can be delivered concurrently (i.e., at the same time) or sequentially.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

The terms “reduce,” “inhibit,” “relieve,” “alleviate” refer to the detectable decrease in symptoms of neuropathic pain, as determined by a trained clinical observer. A reduction in neuropathic pain can be measured by self-assessment (e.g., by reporting of the patient), by applying pain measurement assays well known in the art (e.g., tests for hyperalgesia and/or allodynia), and/or objectively (e.g., using functional magnetic resonance imaging or f-MRI). Determination of a reduction of neuropathic pain can be made by comparing patient status before and after treatment.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than the listed active agents, e.g., an inhibitor of sEHi and/or an EET and an PDEi.

### BRIEF DESCRIPTION OF THE DRAWINGS

FIG. 1. Inhibition of sEH blocks inflammatory and neuropathic pain. (A) Intraspinal administration of the sEHI, AEPU (n=3-4) at low microgram amounts reduced carrageenan elicited peripheral thermal hyperalgesia (black bars, expressed as % control latency) and mechanical allodynia (gray bars, expressed as % control threshold). ANOVA followed by Games-Howell post hoc (*, p=0.012, **p=0.003, ‡, p<0.001). (B) The piperidine sEHI, TPAU (n=6-10) eliminated LPS (i.pl., n=8, 10 μg) elicited thermal hyperalgesia (BL=baseline before LPS) in a dose dependent manner. The metabolically stable TPAU is equipotent to morphine (n=6) but with significantly prolonged efficacy. None of the sEHIs have significant in vitro inhibitory activity on cox-1 or cox-2 (IC₅₀>100 μM, data not shown). (C) Spinal COX2 message is rapidly upregulated following LPS but significantly suppressed by AEPU or TPAU administration (n=6 per group). qRT-PCR measurements reflect fold induction compared to untreated animals in which expression level is set to a value of 1. (D) Brain tissue concentrations of AEPU (n=4) and TPAU (n=4) upon dermal and systemic administration, respectively. (E) TPAU and AUDA two structurally different sEHIs both reduced mechanical allodynia elicited by streptozocin induced diabetic neuropathy (n=6 per group). Allodynia measured by Von Frey's test (BL=baseline withdrawal threshold before streptozocin). Thermal and mechanical withdrawal latencies were converted to percent baseline response and are shown on y-axis. Data are expressed as mean±s.e.m for all figures.

FIG. 2. sEHIs cause a rapid upregulation of spinal StARD1 expression in the presence of elevated intracellular cAMP. (A) In inflamed animals spinal StARD1 mRNA expression was briefly induced in response to peripheral inflammation elicited by LPS (n=4, black bars), but this induction is sustained with AEPU (n=4-5, gray bars) or TPAU (n=4-6, white bars). ANOVA followed by Games-Howell post hoc, *, p=0.03, **, p<0.0001, [symbol] , p=0.04, ♦, p=0.002, ♦♦, p=0.013. (B) In inflamed animals brain StARD1 mRNA expression was only induced in response to LPS+AEPU treatment (n=6, in all groups, * p=0.018, one-way ANOVA followed by Tukey's HSD post hoc) but not by LPS or AEPU alone. (C) In non-inflamed animals direct intraspinal administration of the cell permeable cAMP analogue, 8-Br cAMP (100 μg), methyl esters of EETs (5 μg) and AEPU (1 μg) in saline (with 1% DMSO) led to changes in spinal StARD1 expression after 30 minutes. While saline, AEPU alone, 8-Br cAMP alone did not influence baseline StARD1 levels EETs alone led to a significant decrease. However the combination of cAMP with either EETs of AEPU led to significant increases in spinal StARD1 expression (n=4 for all groups, one-way ANOVA followed by Tukey's HSD post hoc, *, p<0.01). (D) Brain expression levels of StARD1 of animals shown in FIG. 2C were also monitored. In brain slices, only the spinal administration of 8-Br cAMP (100 μg) and AEPU (1 μg) led to a significant increase in brain StARD1 expression. However it is plausible that intraspinal EETs did not reach the brain. Spinal cords and brain slices from saline treated animals were used as calibrators for FIG. 2 C and for FIG. 2 D.

FIG. 3. EET or sEHI mediated antihyperalgesia occurs through two distinct mechanisms. Several cytochrome P450 family enzymes naturally produce EETs by oxidation of the unsaturated bonds of arachidonic acid to result in four regioisomers with pleiotropic biological activities. These are degraded by sEH, which introduces a water molecule opening the epoxide moieties to their corresponding diols or DHETs. The DHETs are widely assumed to be less active. EETs have little effect on the expression of the COX2 gene in normal animals but down regulate induced COX2 possibly through an NF-κB related pathway (Node, et al., Science (1999) 285:1276-1279). Thus increased EETs can mimic anti-inflammatory and analgesic effects of nonsteroidal anti-inflammatory drugs but as transcriptional regulators rather than enzyme inhibitors. EETs also up regulate StARD1 gene expression in the presence of elevated cAMP levels. The StARD1 gene expression leads to an acute increase in steroid/neurosteroid synthesis, which then results in analgesia through an agonistic activity on GABA channels. This results in analgesia in both inflammatory and neuropathic pain states. Paradoxically, COX2 which is repressed by EETs is responsible for producing prostaglandins that through EP receptor activation lead to a rapid rise in intracellular cAMP levels which appear important for EET mediated analgesia. The dashed arrows indicate the novel, hypothesized steps in this cascade.

FIG. 4. The antihyperalgesic effect of AEPU on LPS induced hyperalgesia and the lack of effect of sEHIs and steroid synthesis inhibitors on thermal withdrawal response of rats. (A) AEPU when administered topically into LPS treated animals briefly increased the thermal withdrawal latency (n=8-12). AEPU is rapidly metabolized (B) However, in the absence of inflammation the sEHIs AEPU and TPAU had no effect on thermal withdrawal latency of rats (ANOVA, p=0.19). Animals were administered i.pl. saline (n=6, Δ, 50 ul), topical AEPU (n=6, ▪, 50 mg/kg), and subcutaneous TPAU (n=6, ⋄10 mg/kg).

FIG. 5. Relationship between pain behavior and spinal expression of COX2 and StARD1 genes following LPS induced hyperalgesia. In both plots thermal withdrawal latency vs gene expression levels were graphed by omitting time as a variable. (A) Lack of positive correlation of thermal withdrawal latency (y-axis) and COX2 mRNA levels (x-axis); i.pl. LPS (∘, r²=0.03), AEPU (, r²=0.96) and TPAU (▾, r²=0.83). Percent thermal withdrawal response data from FIG. 1B and FIG. 4 (y-axis) were plotted against gene expression data shown in FIG. 1C (x-axis). Error bars were omitted for clarity. (B) Positive correlation of thermal withdrawal latency (y-axis) and StARD1 mRNA levels (x-axis); i.pl. LPS (▾, r²=0.22), AEPU (, r²=0.99) and TPAU (∘, r²=0.89). Percent thermal withdrawal response data from FIG. 1B and FIG. 4 (y-axis) were plotted against gene expression data shown in FIG. 2A (x-axis); Error bars are omitted for clarity.

FIG. 6. Displacement of TSPO ligand [³H] PK11195 by EETs from the peripheral benzodiazepine receptor (PBR, TSPO). Binding studies were performed by CEREP (France). Data for methyl ester forms of EETs are shown and they did not differ from data generated with the corresponding free acids. No displacement of [³H] flunitrazepam from central benzodiazepine receptors was observed at concentrations up to 100 μM. The 5,6-EET regioisomer was only tested as a methyl ester since the free acid is chemically unstable. The rank order of potency among regioisomers were 14, 15-=5, 6->11, 12->>8,9-EET. The 8,9-EET isomer did not compete with the radioligand and is not shown. Error bars are smaller than the points shown. Data are expressed as mean±s.e.m.

FIG. 7. Steroid synthesis is involved in the antihyperalgesic effect of sEHIs. (A) The general steroid synthesis inhibitor aminoglutethimide (AGL, n=7, 20 mg/kg) or (B) neurosteroid synthesis inhibitor finasteride (FIN, n=8, 20 mg/kg) both blocked the analgesic effect of AEPU (n=6 ∘, 50 mg/kg, topical) on LPS treated rats (n=8). The steroid synthesis inhibitors were administered 30 min prior to LPS. AEPU was administered at the same time as LPS. (C) The steroid synthesis inhibitors aminoglutethimide (topical, n=6, □, 20 mg/kg) and finasteride (topical, n=6, ▪, 20 mg/kg) failed to alter thermal withdrawal latency in control rats treated with only saline (n=6, ∘, 50 μl i.pl.). (D) In LPS administered animals, i.pl. LPS (n=8, , 10 μg) significantly reduced withdrawal latency and topical FIN (n=6, ∘, 20 mg/kg) and AGL (n=6, ▾, 20 mg/kg) co-administered with LPS failed to alter thermal withdrawal latency compared to LPS (ANOVA, p=0.9).

FIG. 8. Effects of inflammation, sEHI and steroid synthesis inhibitor on plasma oxylipins. Inflammation dramatically reduced the EET levels while not changing DHET levels. Expectedly, i.pl. LPS dramatically increased plasma PGE₂ level resulting in pain and inflammation. This increase in PGE₂ was largely restored by treatment with sEHI even in the presence of the antagonist, aminoglutethimide. Sum of quantified sEH substrates (EETs) and products (DHETs) in control (n=5), LPS (n=4), LPS+AEPU (n=5) and LPS+AEPU+AGL (n=4) treated rat plasma. Animals were sampled two hours following inflammation. Black bars, sum of 11, 12- and 14,15-EET ( [symbol] , ANOVA followed by Tukey's HSD post hoc, p=0.01), gray bars, sum of 11, 12- and 14,15-DHET (♦, ANOVA followed by Tukey's HSD post hoc, p<0.003). Dark gray bars, PGE₂ (ANOVA followed by Tukey's HSD post hoc, *, p=0.001, **, p=0.03, ***, p=0.003). Data are expressed as mean±s.e.m.

FIG. 9. Nuclear steroid receptors are not involved in the antihyperalgesic effect of sEHIs. Co-administration of AEPU and antagonists of five nuclear steroid receptors (10 mg/kg each) tamoxifen, TAM for estrogen receptor, mifepristone, MIF for glucocorticoid/progesterone receptor, nilutamide, NIL for androgen receptor and spironolactone, SPR for mineralocorticoid receptor did not block AEPU mediated antihyperalgesia in LPS treated rats (n=4-8, One-way ANOVA, p=0.17).

FIG. 10. Effects of steroid synthesis inhibitors, sEHI and inflammation on circulating hormone levels. (A) Circulating testosterone (black bars) and progesterone (gray bars) levels in control (n=8) and inflamed animals (n=8) two hours following peripheral inflammation. As expected, AGL (n=4), significantly reduced the synthesis of progesterone, ANOVA followed by Games-Howell post hoc (*, p=0.01). (B) Plasma levels of testosterone (n=6, black bar) was not influenced by AEPU administration in either control or LPS treated rats although LPS led to a significant increase in plasma progesterone level (n=6, gray bar). (C) AEPU reduced the mRNA levels of StARD1 induced by ipl. LPS in adrenal glands (gray bars) but not in testis (black bars, n=3-4). Both testis and adrenal gland StARD1 levels were calibrated using spinal levels. ANOVA followed by Games-Howell post hoc, *, p=0.01 adrenal, p=0.16 testis. n.s., not significant. Data are expressed as mean±s.e.m.

FIG. 11. Upregulation of cAMP responsive early genes in the spinal cord following peripheral inflammation. The expression levels of two cAMP responsive genes were used as markers of increase in intracellular levels of cAMP in the spinal cord. Peripheral inflammation led to a time dependent increase in both somatostatin (n=4, black bars) and inducible cAMP early repressor (n=4, gray bars) message levels indicating an increase in intracellular cAMP in these animals. One-way ANOVA followed by Tukey's HSD post hoc, *, p=0.01, **p<0.001, ♦, p=0.01, ♦♦, p<0.001. Data are expressed as mean±s.e.m.

FIG. 12. Antinociceptive effects of rolipram and caffeine and enhancement by sEHi. Data are presented as percent change from each animal's baseline response. A) Rolipram, a selective PDE4 inhibitor, increased thermal withdrawal latencies of rats in a time and dose dependent manner (n=6-12 per dose group). TPAU, a potent sEHi, synergized the effect of rolipram leading to both increased potency (ED₅₀ Rolipram=0.53 mg/kg vs. ED₅₀ Rolipram+TPAU=0.34 mg/kg) and increased efficacy (Rolipram, 202% increase over baseline vs. Rolipram+TPAU, 325% increase over baseline, n=6 for each dose). The sEHi TPAU and AUDA alone had no effect in these assay (see, FIG. 3). B) AUDA, another sEHi, also synergized the effect of rolipram leading to both increased potency (ED₅₀ Rolipram=0.53 mg/kg vs. ED₅₀ Rolipram+AUDA=0.14 mg/kg) and increased efficacy (Rolipram, 202% increase over baseline vs. Rolipram+AUDA, 243% increase over baseline, n=6 per dose). The effect of rolipram+AUDA was antagonized by picrotoxin (0.25 mg/kg). The sEHi+PDEi combinations were not tested at the highest dose of rolipram due to instrumental limitation. C) Rolipram treatment increased mechanical withdrawal threshold of rats in a dose dependent manner (n=6 per dose). This effect was enhanced by AUDA both in potency and efficacy. Picrotoxin antagonized the increase in mechanical withdrawal threshold produced by rolipram+AUDA combination (n=6 per dose). D) Another PDEi, caffeine, also increased mechanical withdrawal threshold of rats in a dose dependent manner (n=6 per dose). Similarly, this effect was enhanced by AUDA. Caffeine at the two lowest doses did not cause depression of motor activity, though its antinociceptive effect was significantly enhanced by AUDA (n=6 per dose). E) Finasteride, a neurosteroid synthesis inhibitor, antagonized the effect of rolipram in a competitive, surmountable manner (n=6 per dose). While celecoxib, a selective cox-2 inhibitor, failed to change the dose-effect curve of rolipram (n=6 per dose). F) Flucanozole, a brain permeable EET synthesis inhibitor, antagonized the effect of rolipram in a non-competitive, non-surmountable manner (n=6 per dose). By contrast miconazole, a brain impermeable EET synthesis inhibitor, failed to change the dose-effect curve of rolipram (n=6 per dose). All compounds were administered by s.c. route.

FIG. 13. Expression of StARD1 and production of allopregnanolone in response to rolipram and sEHi. A) In the rat spinal cord, rolipram led to a biphasic increase in StARD1 expression (n=4 per dose). Co-administration of rolipram and TPAU however led to a dose dependent increase in StARD1 expression (n=4 per dose). B) In the rat brain, rolipram led to no change in StARD1 expression (n=4 per dose). By contrast, co-administration of rolipram and TPAU led to a dose dependent increase in StARD1 expression (n=4 per dose). C) In the rat adrenal gland, TPAU and rolipram both led to a 30% decrease in StARD1 expression (n=4 per dose/treatment). Increasing doses of rolipram however did not further reduce StARD1 expression. Co-administration of rolipram and TPAU slightly increased StARD1 expression however this was a biphasic increase lacking a dose-effect relationship. D) In the rat spinal cord, allopregnanolone levels did not change in response to increasing doses of rolipram (n=4 per dose). However allopregnanolone levels significantly decreased in response to TPAU+rolipram combination (ANOVA, p=0.026). E) In the rat brain, increasing doses of rolipram administration significantly increased allopregnanolone levels at one dose point only (ANOVA, p=0.048). Co-administration of rolipram and TPAU (10 mg/kg, s.c.) did not change brain allopregnanolone levels at any dose point.

FIG. 14. Lack of effect of sEHi alone, blood and brain levels of TPAU and open field activity. A) The two structurally distinct sEHi TPAU and AUDA (n=6 for each group) were administered subcutaneously at indicated doses and thermal withdrawal latencies were monitored over the course of 4 hours. The data are presented as percent change from baseline values. ANOVA analysis revealed no significant differences compared to baseline values (p=0.22 for TPAU and p=0.14 for AUDA). B) Blood and brain inhibitor concentrations of TPAU (10 mg/kg, s.c., n=3 per time point) over the course of 2 hours were quantified. The dashed line indicates the in vitro inhibitory potency (IC₅₀) of TPAU on recombinant rat sEH. TPAU levels both in the blood and brain well exceeded the amount required to inhibit sEH over the course of the experiments. C) The quantification of open field activity demonstrated that TPAU treatment alone did not affect motor function of the animals. However rolipram led to depression of activity which was not significantly different when rolipram and TPAU were co administered at a dose of 0.1 and 10 mg/kg respectively.

FIG. 15. Enhancement of pentobarbital anesthesia and attenuation of picrotoxin induced seizures by sEHi-PDEi combination. A) Pentobarbital administration led of an expected loss of righting response. TPAU or rolipram alone did not change the duration of the loss of righting response. However, the TPAU-rolipram combination significantly increased the duration of the pentobarbital induced loss of righting response. B) Picrotoxin administration (s.c. 10 mg/kg) led to seizure activity with an expected duration to onset. TPAU or rolipram alone did not change the duration to onset of seizures. However, the TPAU-rolipram combination significantly increased the duration to onset of picrotoxin induced seizures. C) In sEH-null mice picrotoxin led to a shorter onset of seizures. However, this was delayed in a dose dependent manner by administration of increasing doses of rolipram.

### DETAILED DESCRIPTION

1. Introduction

We have previously reported that epoxy-eicosatrienoic acids (“EETs”) and inhibitors of the enzyme soluble epoxide hydrolase (“sEH”), or both, are effective in reducing pain when administered systemically or topically. Surprisingly, we have now discovered that the analgesic effect of EETs and sEH inhibitors requires the presence of increased intracellular levels of cyclic adenosine monophosphate (“cAMP”). In light of the findings herein, co-administration of (1) EETs, sEH inhibitors, or both, and (2) agents that increase the intracellular levels of cAMP are useful in providing analgesia and reducing depression in subjects in need thereof. Agents that increase intracellular levels of cAMP are known in the art. In some preferred embodiments, the agents are inhibitors of phosphodiesterase (“PDE”).

It is also known that increased levels of cAMP occur naturally in some disease states. The findings reported herein indicate that inhibition of sEH or increasing levels of EETs, or both, will result in beneficial effects in such conditions. For example, cAMP levels are known to increase during withdrawal from opioids. Administration of sEH inhibitors, or EETs, or both, are therefore expected to ease withdrawal symptoms in patients withdrawing from morphine or heroin. In preferred embodiments, the patient does not have inflammation.

Surprisingly, in the course of the studies reported herein, we also discovered that co-administration of (1) EETs, sEH inhibitors, or both, and (2) agents that increase the intracellular levels of cAMP, delayed onset of seizures in an animal model of epilepsy. On the basis of these studies, we expect that sEH inhibitors, EETs, or both, can be used in combination with commercially available pharmaceutical agents that increase intracellular levels of cAMP to reduce or delay seizures in subjects with epilepsy. In some preferred embodiments, the agents that elevate cAMP are inhibitors of a PDE.

Finally, the results of the studies reported herein surprisingly indicate that sEH inhibitors, EETs, or both, in combination with commercially available pharmaceutical agents that increase intracellular levels of cAMP will be useful in providing post operative analgesia, in which patients need to be kept calm and in reduced pain as they recover from anesthesia. Our studies show in particular that co-administration of an sEH inhibitor and a PDE inhibitor is highly synergistic in producing analgesia and useful for this purpose. In preferred embodiments, the PDE inhibitor is a strong inhibitor, rather than a weak inhibitor such as caffeine. Therapeutically effective amounts of caffeine for purposes of the present invention cannot be obtained by drinking coffee or tea; thus, if the practitioner insists on using caffeine as the PDE inhibitor, it should be administered in a pill or other form more concentrated than caffeine is normally present in coffee or other caffeinated beverages.

2. Patients Subject to Treatment

Generally, the present methods find use in treating a patient who is in a state associated with elevated levels of intracellular cAMP. In patients in a state of elevated levels of intracellular cAMP, co-administration of (i) an epoxygenated fatty acid, e.g., an EET, an inhibitor of sEH or mixtures thereof and (ii) an agent that increases cAMP, e.g., a phosphodiesterase inhibitor, will promote or enhance an analgesic, anesthetic and/or anticonvulsive effect, as needed.

Patients who will benefit from the present methods include those requiring analgesia or anesthesia. The patient may be taking an analgesic or anesthetic agent, e.g., one that elevates intracellular cAMP. The patient may benefit from the reduced dosages of the analgesic or anesthetic that can be administered with the co-administration of the epoxygenated fatty acid (e.g., EET) or inhibitor of sEH and agent that elevates cAMP. In some embodiments, the patient is suffering from inflammatory or neuropathic pain. In some embodiments, the patient has been administered a barbiturate or an opioid receptor agonist. In some embodiments, the patient is recovering from anesthesia.

In some embodiments, the patient suffers from a form of epilepsy, as described herein. In some embodiments, the patient suffers from status epilepticus.

In some embodiments, the patient is experiencing withdrawal from an opioid receptor agonist, e.g., morphine or heroin.

In some embodiment, the patient suffers from depression.

In some embodiments of the invention, the person being treated with EETs, sEHI, or both, does not have hypertension or is not currently being treated with an anti-hypertension agent that is an inhibitor of sEH. In some embodiments, the person being treated does not have inflammation or, if he or she has inflammation, has not been treated with an sEH inhibitor as an anti-inflammatory agent. In some preferred embodiments, the person is being treated for inflammation but by an anti-inflammatory agent, such as a steroid, that is not an inhibitor of sEH. Whether or not any particular anti-inflammatory or anti-hypertensive agent is also an sEH inhibitor can be readily determined by standard assays, such as those taught in U.S. Pat. No. 5,955,496.

In some embodiments, the patient's disease or condition is not caused by an autoimmune disease or a disorder associated with a T-lymphocyte mediated immune function autoimmune response. In some embodiments, the patient does not have a pathological condition selected from type 1 or type 2 diabetes, insulin resistance syndrome, atherosclerosis, coronary artery disease, angina, ischemia, ischemic stroke, Raynaud's disease, or renal disease. In some embodiments, the patient is not a person with diabetes mellitus whose blood pressure is 130/80 or less, a person with metabolic syndrome whose blood pressure is less than 130/85, a person with a triglyceride level over 215 mg/dL, or a person with a cholesterol level over 200 mg/dL or is a person with one or more of these conditions who is not taking an inhibitor of sEH. In some embodiments, the patient does not have an obstructive pulmonary disease, an interstitial lung disease, or asthma. In some embodiments, the patient is not also currently being treated with an inhibitor of one or more enzymes selected from the group consisting of cyclo-oxygenase (“COX”)-1, COX-2, and 5-lipoxygenase (“5-LOX”), or 5-lipoxygenase activating protein (“FLAP”). It is noted that many people take a daily low dose of aspirin (e.g., 81 mg) to reduce their chance of heart attack, or take an occasional aspirin to relieve a headache. It is not contemplated that persons taking low dose aspirin to reduce the risk of heart attack would ordinarily take that aspirin in combination with an EET or sEHI to potentiate that effect. It is also not contemplated that persons taking an occasional aspirin or ibuprofen tablet to relieve a headache or other episodic minor aches or pain would ordinarily take that tablet in combination with an EET or sEHI to potentiate that pain relief, as opposed to persons seeking relief for chronic pain from arthritis or other conditions requiring significant pain relief over an extended period. In some embodiments, therefore, the patient being treated by the methods of the invention may have taken an inhibitor of COX-1, COX-2, or 5-LOX in low doses, or taken such an inhibitor on an occasional basis to relieve an occasional minor ache or pain. In some embodiments, the patient does not have dilated cardiomyopathy or arrhythmia. In some embodiments, the patient is not using EETs or sEHI topically for pain relief. In some embodiments, the patient is not administering EETs or sEHI topically to the eye to relieve, for example, dry eye syndrome or intraocular pressure. In some embodiments, the patient does not have glaucoma or is being treated for glaucoma with agents that do not also inhibit sEH.

3. Epoxygenated Fatty Acids

In some embodiments, an epoxygenated fatty acid is co-administered with an agent that increases intracellular cAMP. Exemplary epoxygenated fatty acids include epoxides of linoleic acid, eicosapentaenoic acid (“EPA”) and docosahexaenoic acid (“DHA”).

The fatty acids eicosapentaenoic acid (“EPA”) and docosahexaenoic acid (“DHA”) have recently become recognized as having beneficial effects, and fish oil tablets, which are a good source of these fatty acids, are widely sold as supplements. In 2003, it was reported that these fatty acids reduced pain and inflammation. Sethi, S. et al., Blood 100: 1340-1346 (2002). The paper did not identify the mechanism of action, nor the agents responsible for this relief.

Cytochrome P450 (“CYP450”) metabolism produces cis-epoxydocosapentaenoic acids (“EpDPEs”) and cis-epoxyeicosatetraenoic acids (“EpETEs”) from docosahexaenoic acid (“DHA”) and eicosapentaenoic acid (“EPA”), respectively. These epoxides are known endothelium-derived hyperpolarizing factors (“EDHFs”). These EDHFs, and others yet unidentified, are mediators released from vascular endothelial cells in response to acetylcholine and bradykinin, and are distinct from the NOS- (nitric oxide) and COX-derived (prostacyclin) vasodilators. Overall cytochrome P450 (CYP450) metabolism of polyunsaturated fatty acids produces epoxides, such as EETs, which are prime candidates for the active mediator(s). 14(15)-EpETE, for example, is derived via epoxidation of the 14,15-double bond of EPA and is the ω-3 homolog of 14(15)-EpETrE (“14(15)EET”) derived via epoxidation of the 14,15-double bond of arachidonic acid.

As mentioned, we have found that it is beneficial to elevate the levels of EETs, which are epoxides of the fatty acid arachidonic acid. Our studies of the effects of EETs has led us to realization that the anti-inflammatory effect of EPA and DHA are likely due to increasing the levels of the epoxides of these two fatty acids. Thus, we expect that increasing the levels of epoxides of EPA, of DHA, or of both, will act to reduce pain and inflammation in mammals in need thereof. This beneficial effect of the epoxides of these fatty acids has not been previously recognized. Moreover, these epoxides have not previously been administered as agents, in part because, as noted above, epoxides have generally been considered too labile to be administered.

Like EETs, the epoxides of EPA and DHA are substrates for sEH. The epoxides of EPA and DHA are produced in the body at low levels by the action of cytochrome P450s. Endogenous levels of these epoxides can be maintained or increased by the administration of sEHI. However, the endogeous production of these epoxides is low and usually occurs in relatively special circumstances, such as the resolution of inflammation. Our expectation is that administering these epoxides from exogenous sources will aid in the resolution of inflammation and in reducing pain. We further expect that it will be beneficial with pain or inflammation to inhibit sEH with sEHI to reduce hydrolysis of these epoxides, thereby maintaining them at relatively high levels.

EPA has five unsaturated bonds, and thus five positions at which epoxides can be formed, while DHA has six. The epoxides of EPA are typically abbreviated and referred to generically as “EpETEs”, while the epoxides of DHA are typically abbreviated and referred to generically as “EpDPEs”. The specific regioisomers of the epoxides of each fatty acid are set forth in the following Table:

TABLE A
Regioisomers of Eicosapentaenoic acid (“EPA”) epoxides:

| | |
|---|---|
| 1. | Formal name: (±)5(6)-epoxy-8Z, 11Z, 14Z, 17Z- |
| | eicosatetraenoic acid, |
| | Synonym 5(6)-epoxy Eicosatetraenoic acid |
| | Abbreviation 5(6)-EpETE |
| 2. | Formal name: (±)8(9)-epoxy-5Z, 11Z, 14Z, 17Z- |
| | eicosatetraenoic acid, |
| | Synonym 8(9)-epoxy Eicosatetraenoic acid |
| | Abbreviation 8(9)-EpETE |
| 3. | Formal name: (±)11(12)-epoxy-5Z, 8Z, 14Z, 17Z- |
| | eicosatetraenoic acid, |
| | Synonym 11(12)-epoxy Eicosatetraenoic acid |
| | Abbreviation 11(12)-EpETE |
| 4. | Formal name: (±)14(15)-epoxy-5Z, 8Z, 11Z, 17Z- |
| | eicosatetraenoic acid, |
| | Synonym 14(15)-epoxy Eicosatetraenoic acid |
| | Abbreviation 14(15)-EpETE |
| 5. | Formal name: (±)17(18)-epoxy-5Z, 8Z, 11Z, 14Z- |
| | eicosatetraenoic acid, |
| | Synonym 17(18)-epoxy Eicosatetraenoic acid |
| | Abbreviation 17(18)-EpETE |

Regioisomers of Docosahexaenoic acid (“DHA”) epoxides:

| | |
|---|---|
| 1. | Formal name: (±) 4(5)-epoxy-7Z, 10Z, 13Z, 16Z, 19Z- |
| | docosapentaenoic acid, |
| | Synonym 4(5)-epoxy Docosapentaenoic acid |
| | Abbreviation 4(5)-EpDPE |
| 2. | Formal name: (±) 7(8)-epoxy-4Z, 10Z, 13Z, 16Z, 19Z- |
| | docosapentaenoic acid, |
| | Synonym 7(8)-epoxy Docosapentaenoic acid |
| | Abbreviation 7(8)-EpDPE |
| 3. | Formal name: (±)10(11)-epoxy-4Z, 7Z, 13Z, 16Z, 19Z- |
| | docosapentaenoic acid, |
| | Synonym 10(11)-epoxy Docosapentaenoic acid |
| | Abbreviation 10(11)-EpDPE |
| 4. | Formal name: (±)13(14)-epoxy-4Z, 7Z, 10Z, 16Z, 19Z- |
| | docosapentaenoic acid, |
| | Synonym 13(14)-epoxy Docosapentaenoic acid |
| | Abbreviation 13(14)-EpDPE |
| 5. | Formal name: (±) 16(17)-epoxy-4Z, 7Z, 10Z, 13Z, 19Z- |
| | docosapentaenoic acid, |
| | Synonym 16(17)-epoxy Docosapentaenoic acid |
| | Abbreviation 16(17)-EpDPE |
| 6. | Formal name: (±) 19(20)-epoxy-4Z, 7Z, 10Z, 13Z, 16Z- |
| | docosapentaenoic acid, |
| | Synonym 19(20)-epoxy Docosapentaenoic acid |
| | Abbreviation 19(20)-EpDPE |

Any of these epoxides, or combinations of any of these, can be administered in the compositions and methods of the invention.

4. Agents that Increase EETs

In some embodiments, an agent that increases intracellular cAMP is co-administered with an agent that increases EETs. Agents that increase EETs include EETs and inhibitors of sEH.

a. Inhibitors of sEH

Scores of sEH inhibitors are known, of a variety of chemical structures. Derivatives in which the urea, carbamate, piperidine or amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U.S. A, 96:8849-8854 (1999), which provides substantial guidance on designing urea derivatives that inhibit the enzyme.

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-admamantyl ureas have been tested and have about the same high activity as an inhibitor of sEH.) Thus, isomers of adamantyl dodecyl urea are preferred inhibitors. It is further expected that N,N′-dodecyl-cyclohexyl urea (DCU), and other inhibitors of sEH, and particularly dodecanoic acid ester derivatives of urea, are suitable for use in the methods of the invention. Preferred inhibitors include:

12-(3-Adamantan-1-yl-ureido)dodecanoic acid (AUDA),

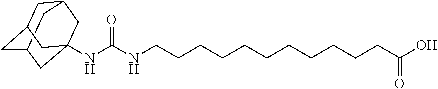

12-(3-Adamantan-1-yl-ureido)dodecanoic acid butyl ester (AUDA-BE),

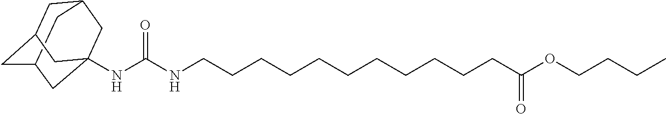

Adamantan-1-yl-3-{5-[2-(2-ethoxyethoxy)ethoxy]pentyl}urea (compound 950, also referred to herein as “AEPU”), and

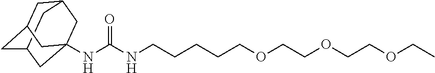

Another preferred group of inhibitors are piperidines. The following Table sets forth some exemplar piperidines and their ability to inhibit sEH activity, expressed as the amount needed to reduce the activity of the enzyme by 50% (expressed as “IC₅₀”).

TABLE 1
IC₅₀ values for selected alkylpiperidine-based sEH inhibitors

| | n = 0 | | n = 1 | |
|---|---|---|---|---|
| 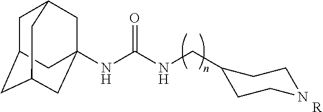 | Compound | IC₅₀ (μM)^{a} | Compound | IC₅₀ (μM)^{a} |
| R: H | I | 0.30 | II | 4.2 |
| 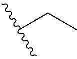 | 3a | 3.8 | 4.a | 3.9 |
| 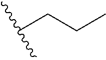 | 3b | 0.81 | 4b | 2.6 |
| 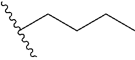 | 3c | 1.2 | 4c | 0.61 |
| 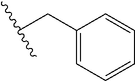 | 3d | 0.01 | 4d | 0.11 |

^{a}As determined via a kinetic fluorescent assay.

A number of other sEH inhibitors which can be used in the methods and compositions of the invention are set forth in co-owned applications PCT/US2008/072199, PCT/US2007/006412, PCT/US2005/038282, PCT/US2005/08765, PCT/US2004/010298 and U.S. Published Patent Application Publication 2005/0026844, each of which is hereby incorporated herein by reference in its entirety for all purposes. U.S. Pat. No. 5,955,496 (the '496 patent) also sets forth a number of sEH inhibitors which can be used in the methods of the invention. One category of these inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S,S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods of the invention are set forth in U.S. Pat. Nos. 6,150,415 (the '415 patent) and 6,531,506 (the '506 patent). Two preferred classes of sEH inhibitors of the invention are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty sEH inhibitors of Formula 2, which were shown to inhibit human sEH at concentrations as low as 0.1 μM. Any particular sEH inhibitor can readily be tested to determine whether it will work in the methods of the invention by standard assays. Esters and salts of the various compounds discussed above or in the cited patents, for example, can be readily tested by these assays for their use in the methods of the invention.

As noted above, chalcone oxides can serve as an alternate substrate for the enzyme. While chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., Medicinal Chemistry: an introduction, John Wiley & Sons Ltd. (West Sussex, England, 2000)). Since the various uses of the invention contemplate inhibition of sEH over differing periods of time which can be measured in days, weeks, or months, chalcone oxides, and other inhibitors which have a half life whose duration is shorter than the practitioner deems desirable, are preferably administered in a manner which provides the agent over a period of time. For example, the inhibitor can be provided in materials that release the inhibitor slowly. Methods of administration that permit high local concentrations of an inhibitor over a period of time are known, and are not limited to use with inhibitors which have short half lives although, for inhibitors with a relatively short half life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., The Pharmacological Basis of Therapeutics, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, the sEH inhibitor used in the methods taught herein is a “soft drug.” Soft drugs are compounds of biological activity that are rapidly inactivated by enzymes as they move from a chosen target site. EETs and simple biodegradable derivatives administered to an area of interest may be considered to be soft drugs in that they are likely to be enzymatically degraded by sEH as they diffuse away from the site of interest following administration. Some sEHI, however, may diffuse or be transported following administration to regions where their activity in inhibiting sEH may not be desired. Thus, multiple soft drugs for treatment have been prepared. These include but are not limited to carbamates, esters, carbonates and amides placed in the sEHI, approximately 7.5 angstroms from the carbonyl of the central pharmacophore. These are highly active sEHI that yield biologically inactive metabolites by the action of esterase and/or amidase. Groups such as amides and carbamates on the central pharmacophores can also be used to increase solubility for applications in which that is desirable in forming a soft drug. Similarly, easily metabolized ethers may contribute soft drug properties and also increase the solubility.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

Preferably, the inhibitor inhibits sEH without also significantly inhibiting microsomal epoxide hydrolase (“mEH”). Preferably, at concentrations of 500 μM, the inhibitor inhibits sEH activity by at least 50% while not inhibiting mEH activity by more than 10%. Preferred compounds have an IC₅₀ (inhibition potency or, by definition, the concentration of inhibitor which reduces enzyme activity by 50%) of less than about 500 μM. Inhibitors with IC₅₀s of less than 500 μM are preferred, with IC₅₀s of less than 100 μM being more preferred and, in order of increasing preference, an IC₅₀ of 50 μM, 40 μM, 30 μM, 25 μM, 20 μM, 15 μM, 10 μM, 5 μM, 3 μM, 2 μM, 1 μM or even less being still more preferred. Assays for determining sEH activity are known in the art and described elsewhere herein.

b. EETs

EETs, which are epoxides of arachidonic acid, are known to be effectors of blood pressure, regulators of inflammation, and modulators of vascular permeability. Hydrolysis of the epoxides by sEH diminishes this activity Inhibition of sEH raises the level of EETs since the rate at which the EETs are hydrolyzed into dihydroxyeicosatrienoic acids (“DHETs”) is reduced.

It has long been believed that EETs administered systemically would be hydrolyzed too quickly by endogenous sEH to be helpful. For example, in one prior report of EETs administration, EETs were administered by catheters inserted into mouse aortas. The EETs were infused continuously during the course of the experiment because of concerns over the short half life of the EETs. See, Liao and Zeldin, International Publication WO 01/10438 (hereafter “Liao and Zeldin”). It also was not known whether endogenous sEH could be inhibited sufficiently in body tissues to permit administration of exogenous EET to result in increased levels of EETs over those normally present. Further, it was thought that EETs, as epoxides, would be too labile to survive the storage and handling necessary for therapeutic use.

Studies from the laboratory of the present inventors, however, showed that systemic administration of EETs in conjunction with inhibitors of sEH had better results than did administration of sEH inhibitors alone. EETs were not administered by themselves in these studies since it was anticipated they would be degraded too quickly to have a useful effect.

Additional studies from the laboratory of the present inventors have since shown, however, that administration of EETs by themselves has had therapeutic effect. Without wishing to be bound by theory, it is surmised that the exogenous EET overwhelms endogenous sEH, and allows EETs levels to be increased for a sufficient period of time to have therapeutic effect. Thus, EETs can be administered without also administering an sEHI to provide a therapeutic effect. Moreover, we have found that EETs, if not exposed to acidic conditions or to sEH are stable and can withstand reasonable storage, handling and administration.

In short, sEHI, EETs, or co-administration of sEHIs and of EETs, can be used in the methods of the present invention. In some embodiments, one or more EETs are administered to the patient without also administering an sEHI. In some embodiments, one or more EETs are administered shortly before or concurrently with administration of an sEH inhibitor to slow hydrolysis of the EET or EETs. In some embodiments, one or more EETs are administered after administration of an sEH inhibitor, but before the level of the sEHI has diminished below a level effective to slow the hydrolysis of the EETs.

EETs useful in the methods of the present invention include 14,15-EET, 8,9-EET and 11,12-EET, and 5,6 EETs. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

If desired, EETs, analogs, or derivatives that retain activity can be used in place of or in combination with unmodified EETs. Liao and Zeldin, supra, define EET analogs as compounds with structural substitutions or alterations in an EET, and include structural analogs in which one or more EET olefins are removed or replaced with acetylene or cyclopropane groups, analogs in which the epoxide moiety is replaced with oxitane or furan rings and heteroatom analogs. In other analogs, the epoxide moiety is replaced with ether, alkoxides, difluorocycloprane, or carbonyl, while in others, the carboxylic acid moiety is replaced with a commonly used mimic, such as a nitrogen heterocycle, a sulfonamide, or another polar functionality. In preferred forms, the analogs or derivatives are relatively stable as compared to an unmodified EET because they are more resistant than an unmodified EET to sEH and to chemical breakdown. “Relatively stable” means the rate of hydrolysis by sEH is at least 25% less than the hydrolysis of the unmodified EET in a hydrolysis assay, and more preferably 50% or more lower than the rate of hydrolysis of an unmodified EET. Liao and Zeldin show, for example, episulfide and sulfonamide EETs derivatives. Amide and ester derivatives of EETs and that are relatively stable are preferred embodiments. In preferred forms, the analogs or derivatives have the biological activity of the unmodified EET regioisomer from which it is modified or derived in binding to the CB2 or peripheral BZD receptor. Whether or not a particular EET analog or derivative has the biological activity of the unmodified EET can be readily determined by using it in standard assays, such as radio-ligand competition assays to measure binding to the relevant receptor. As mentioned in the Definition section, above, for convenience of reference, the term “EETs” as used herein refers to unmodified EETs, and EETs analogs and derivatives unless otherwise required by context.

In some embodiments, the EET or EETs are embedded or otherwise placed in a material that releases the EET over time. Materials suitable for promoting the slow release of compositions such as EETs are known in the art. Optionally, one or more sEH inhibitors may also be placed in the slow release material.

Conveniently, the EET or EETs can be administered orally. Since EETs are subject to degradation under acidic conditions, EETs intended for oral administration can be coated with a coating resistant to dissolving under acidic conditions, but which dissolve under the mildly basic conditions present in the intestines. Suitable coatings, commonly known as “enteric coatings” are widely used for products, such as aspirin, which cause gastric distress or which would undergo degradation upon exposure to gastric acid. By using coatings with an appropriate dissolution profile, the coated substance can be released in a chosen section of the intestinal tract. For example, a substance to be released in the colon is coated with a substance that dissolves at pH 6.5-7, while substances to be released in the duodenum can be coated with a coating that dissolves at pH values over 5.5. Such coatings are commercially available from, for example, Rohm Specialty Acrylics (Rohm America LLC, Piscataway, N.J.) under the trade name “Eudragit®”. The choice of the particular enteric coating is not critical to the practice of the invention.

c. Assays for Epoxide Hydrolase Activity

Any of a number of standard assays for determining epoxide hydrolase activity can be used to determine inhibition of sEH. For example, suitable assays are described in Gill, et al., Anal Biochem 131:273-282 (1983); and Borhan, et al., Analytical Biochemistry 231:188-200 (1995)). Suitable in vitro assays are described in Zeldin et al., J. Biol. Chem. 268:6402-6407 (1993). Suitable in vivo assays are described in Zeldin et al., Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Fla., pp. 303-311 and Wixtrom et al., In: Biochemical Pharmacology and Toxicology, Vol. 1: Methodological Aspects of Drug Metabolizing Enzymes, (Zakim, D. and D. A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, supra, and Hammock. Anal. Biochem. 174:291-299 (1985) and Dietze, et al. Anal. Biochem. 216:176-187 (1994)).

The enzyme also can be detected based on the binding of specific ligands to the catalytic site which either immobilize the enzyme or label it with a probe such as dansyl, fluoracein, luciferase, green fluorescent protein or other reagent. The enzyme can be assayed by its hydration of EETs, its hydrolysis of an epoxide to give a colored product as described by Dietze et al., 1994, supra, or its hydrolysis of a radioactive surrogate substrate (Borhan et al., 1995, supra). The enzyme also can be detected based on the generation of fluorescent products following the hydrolysis of the epoxide. Numerous methods of epoxide hydrolase detection have been described (see, e.g., Wixtrom, supra).

The assays are normally carried out with a recombinant enzyme following affinity purification. They can be carried out in crude tissue homogenates, cell culture or even in vivo, as known in the art and described in the references cited above.

d. Other Means of Inhibiting sEH Activity

Other means of inhibiting sEH activity or gene expression can also be used in the methods of the invention. For example, a nucleic acid molecule complementary to at least a portion of the human sEH gene can be used to inhibit sEH gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet. 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). An exemplary amino acid sequence of human sEH (GenBank Accession No. L05779; SEQ ID NO:1) and an exemplary nucleotide sequence encoding that amino acid sequence (GenBank Accession No. AAA02756; SEQ ID NO:2) are set forth in U.S. Pat. No. 5,445,956. The nucleic acid sequence of human sEH is also published as GenBank Accession No. NM_{—}001979.4; the amino acid sequence of human sEH is also published as GenBank Accession No. NP_{—}001970.2.

A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

For example, using the program available from the Whitehead Institute, the following sEH target sequences and siRNA sequences can be generated:

| | |
|---|---|
| 1) Target: | |
| CAGTGTTCATTGGCCATGACTGG | (SEQ ID NO: 3) |
| Sense-siRNA: | |
| 5′- GUGUUCAUUGGCCAUGACUTT- 3′ | (SEQ ID NO: 4) |
| Antisense-siRNA: | |
| 5′- AGUCAUGGCCAAUGAACACTT- 3′ | (SEQ ID NO: 5) |
| 2) Target: | |
| GAAAGGCTATGGAGAGTCATCTG | (SEQ ID NO: 6) |
| Sense-siRNA: | |
| 5′- AAGGCUAUGGAGAGUCAUCTT- 3′ | (SEQ ID NO: 7) |
| Antisense-siRNA: | |
| 5′- GAUGACUCUCCAUAGCCUUTT- 3′ | (SEQ ID NO: 8) |
| 3) Target | |
| AAAGGCTATGGAGAGTCATCTGC | (SEQ ID NO: 9) |
| Sense-siRNA: | |
| 5′- AGGCUAUGGAGAGUCAUCUTT- 3′ | (SEQ ID NO: 10) |
| Antisense-siRNA: | |
| 5′- AGAUGACUCUCCAUAGCCUTT- 3′ | (SEQ ID NO: 11) |
| 4) Target: | |
| CAAGCAGTGTTCATTGGCCATGA | (SEQ ID NO: 12) |
| Sense-siRNA: | |
| 5′- AGCAGUGUUCAUUGGCCAUTT- 3′ | (SEQ ID NO: 13 |
| Antisense-siRNA: | |
| 5′- AUGGCCAAUGAACACUGCUTT- 3′ | (SEQ ID NO: 14 |
| 5) Target: | |
| CAGCACATGGAGGACTGGATTCC | (SEQ ID NO: 15) |
| Sense-siRNA: | |
| 5′- GCACAUGGAGGACUGGAUUTT- 3′ | (SEQ ID NO: 16) |
| Antisense-siRNA: | |
| 5′- AAUCCAGUCCUCCAUGUGCTT- 3′ | (SEQ ID NO: 17) |

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 by siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 by dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplar vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO:18). Further, 5-6 T′ s are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. To attack the same targets by short hairpin RNAs, produced by a vector (permanent RNAi effect), sense and antisense strand can be put in a row with a loop forming sequence in between and suitable sequences for an adequate expression vector to both ends of the sequence. The following are non-limiting examples of hairpin sequences that can be cloned into the pSuper vector:

1) Target:
(SEQ ID NO: 19)
CAGTGTTCATTGGCCATGACTGG
Sense strand:
(SEQ ID NO: 20)
5′-GATCCCCGTGTTCATTGGCCATGACTTTCAA
GAGAAGTCATGGCCAATGAACACTTTTT-3′
Antisense strand:
(SEQ ID NO: 21)
5′-AGCTAAAAAGTGTTCATTGGCCATGACTTCTCTT
GAAAGTCATGGCCAATGAACACGGG -3′
2) Target:
(SEQ ID NO: 22)
GAAAGGCTATGGAGAGTCATCTG
Sense strand:
(SEQ ID NO: 23)
5′-GATCCCCAAGGCTATGGAGAGTCATCTTCAAGAGAGA
TGACTCTCCATAGCCTTTTTTT -3′
Antisense strand:
(SEQ ID NO: 24)
5′- AGCTAAAAAAAGGCTATGGAGAGTCATCTCTCTTGAA
GATGACTCTCCATAGCCTTGGG -3′
3) Target:
(SEQ ID NO: 25)
AAAGGCTATGGAGAGTCATCTGC
Sense strand:
(SEQ ID NO: 26)
5′-GATCCCCAGGCTATGGAGAGTCATCTTTCAAGAGAAG
ATGACTCTCCATAGCCTTTTTT -3′
Antisense strand:
(SEQ ID NO: 27)
5′-AGCTAAAAAAGGCTATGGAGAGTCATCATCTCTTGAAAGATGACTCT
CCATAGCCTGGG -3′
4) Target:
(SEQ ID NO: 28)
CAAGCAGTGTTCATTGGCCATGA
Sense strand:
(SEQ ID NO: 29)
5′-GATCCCCAGCAGTGTTCATTGGCCATTTCAAGAGAATG
GCCAATGAACACTGCTTTTTT -3′
Antisense strand:
(SEQ ID NO: 30)
5′- AGCTAAAAAAGCAGTGTTCATTGGCCATTCTCTTGAAATG
GCCAATGAACACTGCTGGG -3′
5) Target:
(SEQ ID NO: 31)
CAGCACATGGAGGACTGGATTCC
Sense strand
(SEQ ID NO: 32)
5′-GATCCCCGCACATGGAGGACTGGATTTTCAAGAGAAATC
CAGTCCTCCATGTGCTTTTT -3′
Antisense strand:
(SEQ ID NO: 33)
5′-AGCTAAAAAGCACATGGAGGACTGGATTTCTCTTGAAAA
TCCAGTCCTCCATGTGCGGG -3′

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. In one embodiment, the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program found on the worldwide web “biotools.idtdna.com/antisense/AntiSense.aspx”, which will provide appropriate antisense sequences for nucleic acid sequences up to 10,000 nucleotides in length. Using this program with the sEH gene provides the following exemplar sequences:

| | | |
|---|---|---|
| 1) | UGUCCAGUGCCCACAGUCCU | (SEQ ID NO: 34) |
| 2) | UUCCCACCUGACACGACUCU | (SEQ ID NO: 35) |
| 3) | GUUCAGCCUCAGCCACUCCU | (SEQ ID NO: 36) |
| 4) | AGUCCUCCCGCUUCACAGA | (SEQ ID NO: 37) |
| 5) | GCCCACUUCCAGUUCCUUUCC | (SEQ ID NO: 38) |

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a sEH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996). Another subsequence, the h (hydrophobic) domain of signal peptides, was found to have similar cell membrane translocation characteristics (see, e.g., Lin et al., J. Biol. Chem. 270:14255-14258 (1995)). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

More recently, it has been discovered that siRNAs can be introduced into mammals without eliciting an immune response by encapsulating them in nanoparticles of cyclodextrin. Information on this method can be found on the worldwide web at “nature.com/news/2005/050418/full/050418-6.html.”

In another method, the nucleic acid is introduced directly into superficial layers of the skin or into muscle cells by a jet of compressed gas or the like. Methods for administering naked polynucleotides are well known and are taught, for example, in U.S. Pat. No. 5,830,877 and International Publication Nos. WO 99/52483 and 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus.

miRNAs and siRNAs differ in several ways: miRNA derive from points in the genome different from previously recognized genes, while siRNAs derive from mRNA, viruses or transposons, miRNA derives from hairpin structures, while siRNA derives from longer duplexed RNA, miRNA is conserved among related organisms, while siRNA usually is not, and miRNA silences loci other than that from which it derives, while siRNA silences the loci from which it arises. Interestingly, miRNAs tend not to exhibit perfect complementarity to the mRNA whose expression they inhibit. See, McManus et al., supra. See also, Cheng et al., Nucleic Acids Res. 33(4):1290-7 (2005); Robins and Padgett, Proc Natl Acad Sci USA. 102(11):4006-9 (2005); Brennecke et al., PLoS Biol. 3(3):e85 (2005). Methods of designing miRNAs are known. See, e.g., Zeng et al., Methods Enzymol. 392:371-80 (2005); Krol et al., J Biol. Chem. 279(40):42230-9 (2004); Ying and Lin, Biochem Biophys Res Commun. 326(3):515-20 (2005).

5. Agents that Increase Cyclic AMP

The agents that increase EETs can be co-administered with an agent that increases intracellular cyclic AMP (cAMP), i.e., a cAMP elevating agent. cAMP elevating agents are known in the art and include agents that activate adenylate cyclase, agents that inhibit a cAMP phosphodiesterase, and cAMP analogs.

In some embodiments, the agent that increases cAMP is an activator of adenylate cyclase. Exemplary agents that activate adenylate cyclase include forskolin, prostaglandin E2, and pituitary adenylate cyclase activating peptide (PACAP).

In some embodiments, the agent that increases cAMP is an inhibitor of a cAMP phosphodiesterase (PDE), e.g., a cyclic nucleotide phosphodiesterases (PDE) that degrades the phosphodiester bond in the second messenger molecules cAMP. The inhibitor may or may not be selective, specific or preferential for cAMP. Exemplary PDEs that degrade cAMP include without limitation PDE3, PDE4, PDE7, PDE8 and PDE10. Exemplary cAMP selective hydrolases include PDE4, 7 and 8. Exemplary PDEs that hydrolyse both cAMP and cGMP include PDE1, 2, 3, 10 and 11. Isoenzymes and isoforms of PDEs are well known in the art. See, e.g., Boswell-Smith et al., “Phosphoediesterase inhibitors”, Brit. J. Pharmacol. 147:5252-257 (2006), and Reneerkens, et al., Psychopharmacology (2009) 202:419-443, the contents of which are incorporated herein by reference.

In some embodiments, the PDE inhibitor is a non-selective inhibitor of PDE. Exemplary non-selective PDE inhibitors that find use include without limitation caffeine, theophylline, isobutylmethylxanthine, aminophylline, pentoxifylline, vasoactive intestinal peptide (VIP), secretin, adrenocorticotropic hormone, pilocarpine, alpha-melanocyte stimulating hormone (MSH), beta-MSH, gamma-MSH, the ionophore A23187, prostaglandin E1.

In some embodiments, the PDE inhibitor used specifically or preferentially inhibits PDE4. Exemplary inhibitors that selectively inhibit PDE4 include without limitation rolipram, roflumilast, cilomilast, ariflo, HT0712, ibudilast and mesembrine.

In some embodiments, the PDE inhibitor used specifically or preferentially inhibits a cAMP PDE, e.g., PDE4, PDE7 or PDE8. In some embodiments, the PDE inhibitor used inhibits a cAMP PDE, e.g., PDE1, PDE2, PDE3, PDE4, PDE7, PDE8, PDE10 or PDE11. Exemplary agents that inhibit a cAMP phosphodiesterase include without limitation rolipram, roflumilast, cilomilast, ariflo, HT0712, ibudilast, mesembrine, cilostamide, enoxamone, milrinone, siguazodan and BRL-50481.

In some embodiments, the PDE inhibitor used specifically inhibits PDE5. Exemplary inhibitors that selectively inhibit PDE5 include without limitation sildenafil, zaprinast, tadalafil, udenafil, avanafil and vardenafil.

In some embodiments, the agent that increases cAMP is a cAMP analog. Exemplary cAMP analogs include without limitation dibutyryl adenosine 3′,5′-cyclic monophosphate (DBcAMP), 8-(4-chlorophenylthio)-cAMP, 8-bromo cAMP, N⁶ benzoyl cAMP.

6. Co-Administration of an Agent that Increases EETs or Epoxygenated Fatty Acid with an Agent that Increases cAMP

The epoxygenated fatty acid or agent that increases EETs (e.g., EETs, inhibitors of sEH) and the agent that increases cAMP (e.g., cAMP, inhibitors of PDE) can be prepared and administered independently or together in a wide variety of oral, parenteral and aerosol formulations. In some preferred forms, compounds for use in the methods of the present invention can be administered by injection, that is, intravenously, intramuscularly, intracutaneously, subcutaneously, intradermally, topically, intraduodenally, or intraperitoneally, while in others, they are administered orally. Administration can be systemic or local, as desired. The epoxygenated fatty acid or agent that increases EETs or the agent that increases cAMP, or all co-administered agents, can also be administered by inhalation. Additionally, the epoxygenated fatty acid or agent that increases EETs or the agent that increases cAMP, or all co-administered agents, can be administered transdermally. Accordingly, the methods of the invention permit administration of pharmaceutical compositions comprising a pharmaceutically acceptable carrier or excipient and either a selected inhibitor or a pharmaceutically acceptable salt of the inhibitor.

For preparing pharmaceutical compositions from an epoxygenated fatty acid or agent that increases EETs or the agent that increases cAMP, or all co-administered agents, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

A variety of solid, semisolid and liquid vehicles have been known in the art for years for topical application of agents to the skin. Such vehicles include creams, lotions, gels, balms, oils, ointments and sprays. See, e.g., Provost C. “Transparent oil-water gels: a review,” Int J Cosmet Sci. 8:233-247 (1986), Katz and Poulsen, Concepts in biochemical pharmacology, part I. In: Brodie B B, Gilette J R, eds. Handbook of Experimental Pharmacology. Vol. 28. New York, N.Y.: Springer; 107-174 (1971), and Hadgcraft, “Recent progress in the formulation of vehicles for topical applications,” Br J. Dermatol., 81:386-389 (1972). A number of topical formulations of analgesics, including capsaicin (e.g., Capsin®), so-called “counter-irritants” (e.g., Icy-Hot®, substances such as menthol, oil of wintergreen, camphor, or eucalyptus oil compounds which, when applied to skin over an area presumably alter or off-set pain in joints or muscles served by the same nerves) and salicylates (e.g. BenGay®), are known and can be readily adapted for topical administration of the epoxygenated fatty acid or agent that increases EETs or the agent that increases cAMP, or all co-administered agents, by replacing the active ingredient or ingredient with an epoxygenated fatty acid or agent that increases EETs or the agent that increases cAMP, or all co-administered agents. It is presumed that the person of skill is familiar with these various vehicles and preparations and they need not be described in detail herein.

The epoxygenated fatty acid or agent that increases EETs or the agent that increases cAMP, or all co-administered agents, can be mixed into such modalities (creams, lotions, gels, etc.) for topical administration. In general, the concentration of the agents provides a gradient which drives the agent into the skin. Standard ways of determining flux of drugs into the skin, as well as for modifying agents to speed or slow their delivery into the skin are well known in the art and taught, for example, in Osborne and Amann, eds., Topical Drug Delivery Formulations, Marcel Dekker, 1989. The use of dermal drug delivery agents in particular is taught in, for example, Ghosh et al., eds., Transdermal and Topical Drug Delivery Systems, CRC Press, (Boca Raton, Fla., 1997).

In some embodiments, the agents are in a cream. Typically, the cream comprises one or more hydrophobic lipids, with other agents to improve the “feel” of the cream or to provide other useful characteristics. In one embodiment, for example, a cream of the invention may contain 0.01 mg to 10 mg of sEHI, with or without one or more EETs, per gram of cream in a white to off-white, opaque cream base of purified water USP, white petrolatum USP, stearyl alcohol NF, propylene glycol USP, polysorbate 60 NF, cetyl alcohol NF, and benzoic acid USP 0.2% as a preservative. In the studies reported in the Examples, sEHI were mixed into a commercially available cream, Vanicream® (Pharmaceutical Specialties, Inc., Rochester, Minn.) comprising purified water, white petrolatum, cetearyl alcohol and ceteareth-20, sorbitol solution, propylene glycol, simethicone, glyceryl monostearate, polyethylene glycol monostearate, sorbic acid and BHT.

In other embodiments, the agent or agents are in a lotion. Typical lotions comprise, for example, water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, tri (PPG-3 myristyl ether) citrate, disodium EDTA, methylparaben, ethylparaben, propylparaben, xanthan gum, butylparaben, and methyldibromo glutaronitrile.

In some embodiments, the agent is, or agents are, in an oil, such as jojoba oil. In some embodiments, the agent is, or agents are, in an ointment, which may, for example, white petrolatum, hydrophilic petrolatum, anhydrous lanolin, hydrous lanolin, or polyethylene glycol. In some embodiments, the agent is, or agents are, in a spray, which typically comprise an alcohol and a propellant. If absorption through the skin needs to be enhanced, the spray may optionally contain, for example, isopropyl myristate.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the epoxygenated fatty acid, the sEH inhibitor, the EETs, or all co-administered agents, is employed in reducing, alleviating, relieving, ameliorating, preventing and/or inhibiting neuropathic pain. The dosage of the specific compound for treatment depends on many factors that are well known to those skilled in the art. They include for example, the route of administration and the potency of the particular compound.

Determination of an effective amount is well within the capability of those skilled in the art. Generally, an efficacious or effective amount of an epoxygenated fatty acid or agent that increases EET or the agent that increases cAMP, or all co-administered agents, is determined by first administering a low dose or a small amount of either the epoxygenated fatty acid or agent that increases EETs or the agent that increases cAMP, or all co-administered agents, and then incrementally increasing the administered dose or dosages, adding a second medication as needed, until a desired effect of is observed in the treated subject with minimal or no toxic side effects. An exemplary dose of an sEHi or EET is from about 0.001 μM/kg to about 100 mg/kg body weight of the mammal. sEH inhibitors with lower IC50 concentrations can be administered in lower doses.

Efficacious doses of phosphodiesterase inhibitors and neurosteroids are also known in the art. The present invention utilizes doses that are equivalent or less, e.g., doses that are about 75%, 50% or 25% of a full dose, to those prescribed for these agents when they are not co-administered with an epoxygenated fatty acid, EET or inhibitor of sEHi. See, e.g., Physicians' Desk Reference 2009 (PDR, 63rd Edition) by Physicians' Desk Reference, 2008, Thomson Reuters.

EETs are unstable in acidic conditions, and can be converted to DHETs. To avoid conversion of orally administered EETs to DHETs under the acidic conditions present in the stomach, EETs can be administered intravenously, by injection, or by aerosol. EETs intended for oral administration can be encapsulated in a coating that protects the EETs during passage through the stomach. For example, the EETs can be provided with a so-called “enteric” coating, such as those used for some brands of aspirin, or embedded in a formulation. Such enteric coatings and formulations are well known in the art. In some formulations, the epoxygenated fatty acid or agent that increases EETs or the agent that increases cAMP, or all co-administered agents, are embedded in a slow-release formulation to facilitate administration of the agents over time.

In another set of embodiments, the epoxygenated fatty acid or agent that increases EETs or the agent that increases cAMP, or all co-administered agents, are administered by delivery to the nose or to the lung. Intranasal and pulmonary delivery are considered to be ways drugs can be rapidly introduced into an organism. Devices for delivering drugs intranasally or to the lungs are well known in the art. The devices typically deliver either an aerosol of an therapeutically active agent in a solution, or a dry powder of the agent. To aid in providing reproducible dosages of the agent, dry powder formulations often include substantial amounts of excipients, such as polysaccharides, as bulking agents.

Detailed information about the delivery of therapeutically active agents in the form of aerosols or as powders is available in the art. For example, the Center for Drug Evaluation and Research (“CDER”) of the U.S. Food and Drug Administration provides detailed guidance in a publication entitled: “Guidance for Industry: Nasal Spray and Inhalation Solution, Suspension, and Spray Drug Products—Chemistry, Manufacturing, and Controls Documentation” (Office of Training and Communications, Division of Drug Information, CDER, FDA, July 2002). This guidance is available in written form from CDER, or can be found on the worldwide web at “fda.gov/cder/guidance/4234fn1.htm”. The FDA has also made detailed draft guidance available on dry powder inhalers and metered dose inhalers. See, Metered Dose Inhaler (MDI) and Dry Powder Inhaler (DPI) Drug Products—Chemistry, Manufacturing, and Controls Documentation, 63 Fed. Reg. 64270, (November 1998). A number of inhalers are commercially available, for example, to administer albuterol to asthma patients, and can be used instead in the methods of the present invention to administer the epoxygenated fatty acid or agent that increases EET or the agent that increases cAMP, or both of the two agents to subjects in need thereof.

In some aspects of the invention, the epoxygenated fatty acid or agent that increases EET or the agent that increases cAMP, or all co-administered agents, is dissolved or suspended in a suitable solvent, such as water, ethanol, or saline, and administered by nebulization. A nebulizer produces an aerosol of fine particles by breaking a fluid into fine droplets and dispersing them into a flowing stream of gas. Medical nebulizers are designed to convert water or aqueous solutions or colloidal suspensions to aerosols of fine, inhalable droplets that can enter the lungs of a patient during inhalation and deposit on the surface of the respiratory airways. Typical pneumatic (compressed gas) medical nebulizers develop approximately 15 to 30 microliters of aerosol per liter of gas in finely divided droplets with volume or mass median diameters in the respirable range of 2 to 4 micrometers. Predominantly, water or saline solutions are used with low solute concentrations, typically ranging from 1.0 to 5.0 mg/mL.

Nebulizers for delivering an aerosolized solution to the lungs are commercially available from a number of sources, including the AERx™ (Aradigm Corp., Hayward, Calif.) and the Acorn II® (Vital Signs Inc., Totowa, N.J.).

Metered dose inhalers are also known and available. Breath actuated inhalers typically contain a pressurized propellant and provide a metered dose automatically when the patient's inspiratory effort either moves a mechanical lever or the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978; and 4,896,832.

The formulations may also be delivered using a dry powder inhaler (DPI), i.e., an inhaler device that utilizes the patient's inhaled breath as a vehicle to transport the dry powder drug to the lungs. Such devices are described in, for example, U.S. Pat. Nos. 5,458,135; 5,740,794; and 5,785,049. When administered using a device of this type, the powder is contained in a receptacle having a puncturable lid or other access surface, preferably a blister package or cartridge, where the receptacle may contain a single dosage unit or multiple dosage units.

Other dry powder dispersion devices for pulmonary administration of dry powders include those described in Newell, European Patent No. EP 129985; in Hodson, European Patent No. EP 472598, in Cocozza, European Patent No. EP 467172, and in Lloyd, U.S. Pat. Nos. 5,522,385; 4,668,281; 4,667,668; and 4,805,811. Dry powders may also be delivered using a pressurized, metered dose inhaler (MDI) containing a solution or suspension of drug in a pharmaceutically inert liquid propellant, e.g., a chlorofluorocarbon or fluorocarbon, as described in U.S. Pat. Nos. 5,320,094 and 5,672,581.

Without further elaboration, it is believed that one skilled in the art can, using the preceding description, practice the present invention to its fullest extent.

7. Kits

The pharmaceutical compositions of the present invention can be provided in a kit. Generally, the kits comprise (i) an epoxygenated fatty acid or an agent that increases EETs (e.g., an sEHi or an EET, or both) and (ii) an agent that increases cAMP (e.g., a PDEi).

In certain embodiments, a kit of the present invention comprises the epoxygenated fatty acid or the agent that increases EETs (e.g., an sEHi or an EET, or both) or the agent that increases cAMP (e.g., a PDEi) in separate formulations. In certain embodiments, the kits comprise the epoxygenated fatty acid or the agent that increases EETs (e.g., an sEHi or an EET, or both) or the agent that increases cAMP (e.g., a PDEi) within the same formulation. In certain embodiments, the kits provide the epoxygenated fatty acid or agent that increases EETs (e.g., an sEHi or an EET, or both) or the agent that increases cAMP (e.g., a PDEi) in uniform dosage formulations throughout the course of treatment. In certain embodiments, the kits provide the epoxygenated fatty acid or agent that increases EETs (e.g., an sEHi or an EET, or both) or the agent that increases cAMP (e.g., a PDEi) in graduated dosages over the course of treatment, either increasing or decreasing, but usually increasing to an efficacious dosage level, according to the requirements of an individual.

Further embodiments of the kits are as described herein. In some embodiments, the epoxygenated fatty acid is an epoxy-eicosatrienoic acid (“EET”). Exemplary EETs that find use include 14,15-EET, 8,9-EET and 11,12-EET, and 5,6 EETs.

In some embodiments, the epoxygenated fatty acid is an epoxide of linoleic acid, eicosapentaenoic acid (“EPA”) or docosahexaenoic acid (“DHA”), or a mixture thereof.

In some embodiments, the agent that increases intracellular levels of cAMP is an inhibitor of phosphodiesterase. In some embodiments, the inhibitor of phosphodiesterase is a non-selective inhibitor of phosphodiesterase. In some embodiments, the inhibitor of phosphodiesterase selectively inhibits a cAMP phosphodiesterase isozyme, for example, PDE3, PDE4, PDE7, PDE8 and PDE10.

In some embodiments, the inhibitor of phosphodiesterase is an inhibitor of PDE4. Exemplary inhibitors of PDE4 that find use include without limitation, rolipram, roflumilast, cilomilast, ariflo, HT0712, ibudilast, mesembrine, pentoxifylline, piclamilast, and combinations thereof. In some embodiments, the inhibitor of phosphodiesterase is rolipram.

In some embodiments, the inhibitor of phosphodiesterase is an inhibitor of PDE5. Exemplary inhibitors of PDE5 that find use include without limitation, sildenafil, zaprinast, tadalafil, vardenafil and combinations thereof.

The kits may also provide instructions for use, for example, for reducing the frequency and/or duration of seizures or for enhancing anesthesia or analgesia in a subject in need thereof.

### EXAMPLES

The following examples are offered to illustrate, but not to limit the claimed invention.

### Example 1

### Soluble Epoxide Hydrolase and Epoxyeicosatrienoic Acids Modulate a Classical and a Novel Analgesic Pathway

During inflammation, a large amount of arachidonic acid (AA) is released into the cellular milieu and cyclooxygenase enzymes convert this AA to prostaglandins that in turn sensitize pain pathways. However, AA is also converted to natural epoxyeicosatrienoic acids (EETs) by cytochrome P450 enzymes. EET levels are typically regulated by soluble epoxide hydrolase (sEH), the major enzyme degrading EETs. Here we demonstrate that EETs or inhibition of sEH lead to antihyperalgesia by at least two spinal mechanisms, firstly, by repressing the induction of the COX2 gene and secondly, by rapidly upregulating an acute neurosteroid producing gene, StARD1, which requires the synchronized presence of elevated cAMP and EET levels. The analgesic activities of neurosteroids are well known however here we describe a clear course towards augmenting the levels of these molecules. Redirecting the flow of pro-nociceptive intracellular cAMP towards upregulation of StARD1 mRNA by concomitantly elevating EETs is a novel path to accomplish pain relief in both inflammatory and neuropathic pain states.

### Materials and Methods

### Animals, Treatments Pain Models and Nociceptive Testing

The study was approved by UC Davis Animal Care and Use Committee. Male Sprague-Dawley rats weighing 250-350 g were obtained from Charles River Inc. Two models of inflammatory pain and one model of diabetic neuropathic pain were used to test the effects of sEHI. The main inflammatory pain model used involved intraplantar LPS (10 μg/animal) administration as described previously (Inceoglu B et al., Life Sci 79:2311-2319 (2006)). Briefly, following baseline thermal withdrawal latency and mechanical withdrawal threshold determination LPS (in saline) was administered into one hind paw and nociceptive thresholds were monitored over time. The other inflammatory pain model involved intraplantar carrageenan (1% in saline, 50 μl) administration and was only used when sEHI was administered into the spinal cord. Intrathecal catheters were implanted according to Yaksh and Rudy (Yaksh T L and Rudy T A, Physiol Behav 17(6):1031-1036 (1976)). Following baseline nociceptive response measurements carrageenan was administered into one hind paw and responses were monitored over time and after intraspinal sEHI administration. In experiments in which steroid synthesis inhibitors and steroid receptor antagonists were used, the antagonist compounds were administered topically one hour prior to testing Inhibitors of sEH were administered topically as described, or dissolved in trans free trioleate and given subcutaneously or dissolved in sterile saline and given through intrathecal catheters as indicated. Morphine was dissolved in sterile saline and administered subcutaneously to LPS injected animals. Diabetic neuropathy was induced as described by Aley and Levine (Aley K O and Levine J D, J Pain 2:146-150 (2001)). Thermal withdrawal latencies and mechanical withdrawal thresholds were corrected to baseline responses and reported as percent control latency or threshold as described previously (Inceoglu B et al., Life Sci 79:2311-2319 (2006)). In experiments in which EETs, sEHI and cAMP analogue was administered intraspinally animals were maintained under deep anesthesia and therefore nociceptive thresholds were not determined.

### Oxylipin Analysis

Oxylipins were analyzed as described previously (Schmelzer K et al., Proc Natl Acad Sci USA 102:9772-9777 (2005)).

### Quantitative Real Time RT-PCR

A purelink Micro to Midi total RNA purification kit (Invitrogen, CA) was used to extract RNA from whole spinal cord, adrenal gland and testis. For brain, the remaining caudal two thirds were sliced into two hemispheres from the midline and a coronal slice of 2 mm in thickness was taken for analysis. The RNA samples were quantified by spectrophotometry and converted to cDNA using a high capacity cDNA reverse transcription kit from Applied Biosystems (CA, USA). Taq-man probes for COX2 (Rn00568225_ml) StARD1 (Rn00580695_ml), ICER(Rn00569145_ml), Sst (Rn00561967_ml), sEH(Rn00576023_ml) genes were used according to manufacturer's instructions to quantify relative gene expression (Applied Biosystems). Experiments were performed in triplicate with GAPDH (glyceraldehyde 3-phosphate dehydrogenase message) serving as the endogenous control. Mean fold expression values from corresponding untreated animal tissues were used as calibrators.

The increase in spinal intracellular cAMP upon LPS elicited inflammation was assessed by monitoring the expression of ICER, (inducible cAMP early repressor) an immediate early transcription factor, as well as somatostatin gene both of which are known to respond rapidly to a rise in intracellular cAMP levels (Bodor J et al., Proc Natl Acad Sci USA 93:3536-3541 (1996)).

To investigate the cooperation between cAMP and EETs in the spinal cord, a membrane permeable cAMP analogue, 8-Br cAMP (100 μg), was administered into the spinal cord by lumbar puncture (between L4-L5) and rapid (30 min.) expression of StARD1 mRNA in the spinal cord and the brain was quantified by qRT-PCR. In these experiments animals were maintained under deep anesthesia.

### Supporting Methods

### Receptor Binding and Hormone Assays

Binding assays were performed by CEREP (France) on a contract basis using the procedures of Le Fur (Le Fur G et al., Life Sci 32:1839-1847 (1983)) and Speth (Speth R C et al., Life Sci 24:351-357 (1979)) with [³H] PK11195 and rat heart mitochondria for TSPO assays and [³H] flunitrazepam and rat cerebral cortex for central benzodiazepine receptors. EET methyl esters were synthesized as described (Campbell W et al., Endocrinology 128:2183-2194 (1991)). Steroid hormones were assayed by RIA at UC Davis, Clinical Endocrinology Laboratory according to standard procedures.

### Tissue Sampling

Plasma was collected by cardiac puncture under deep anesthesia. Rats were anesthetized by isoflurane overdose and decapitated. Whole brains were immediately excised and flash frozen. Spinal cord was rapidly removed following a full laminectomy of the regions between L1-L5. Dorsal roots were excluded. Adrenal glands and testis from the same animals were removed, flash frozen and stored at −80° C.

### Enzyme Assay and Chemical Synthesis

Soluble epoxide hydrolase activity was determined by a modification of a procedure described previously (Wixtrom R N and Hammock B D, Anal Biochem 174:291-299 (1988)). Recombinant human, mouse, and rat sEH enzymes were produced in a baculovirus expression system and purified by affinity chromatography (Wixtrom R N and Hammock B D, Anal Biochem 174:291-299 (1988)). Protein concentration was quantified using the Pierce BCA assay with bovine serum albumin (BSA) as calibrating standard. The concentration of inhibitor that reduces enzyme activity by 50% was designated as IC₅₀. For the human, mouse and rat sEH, the IC₅₀s for inhibitors were determined using cyano(2-methoxynaphthalen-6-yl)methyl trans-(3-phenyl-oxyran-2-yl) methyl carbonate as a fluorescent substrate (Jones P D et al., Anal Biochem 343):66-75 (2005)). The calculation of IC₅₀s were based on regression equations composed of at least five datum points with a minimum of two points in the linear region of the curve on either side of the IC₅₀. Results are average of three experiments. Inhibitors of sEH were synthesized, purified and characterized in our laboratory as described previously (Jones P D et al., Bioorg Med Chem Lett 16:5212-5216 (2006); Morisseau C and Hammock B D, in Techniques for analysis of chemical biotransormation, Current Protocols in Toxicology, eds Bus J S, Costa L G, Hodgson E, Lawrence D A, Reed D J (John Wiley & Sons, New Jersey), pp 4.23.1-18 (2007).). EETs and methyl ester analogues of EETs were synthesized as described (Campbell W et al., Endocrinology 128:2183-2194 (1991)).

### Brain Inhibitor Level Analysis

Prefrontal cortex (rostral one third) of the brain was separated from frozen brain tissue with a coronal cut. A section of approximately 2×2×2 mm was then removed from the core of this region. All other procedures were performed on ice or at 4° C. Ten μl of a surrogate internal and extraction standard (compound 869, 1-adamantan-1-yl-3-(5-butoxy-pentyl)-urea, 250 ng/ml) was added to each sample prior to homogenization and centrifugation (10,000×g) for 5 min in 1 ml ethyl acetate. This step was repeated two more times and supernatants were pooled. These samples were then evaporated using a vacuum centrifuge, reconstituted in 50 μl of compound 790 (1-adamantan-1-yl-3-(12-imidazole-1-yl-dodecyl)-urea, 50 ng/ml) as internal standard, filtered through a 0.1 μm PVDF membrane (Millipore, Billerica, Mass.) and stored frozen. LC-ESI-MS/MS analysis was performed using a HPLC separation module (Waters, Milford, Mass.) interfaced to a Quattro Premier triple-quadrupole mass spectrometer (Waters) operating in positive electrospray ionization mode with multiple reaction monitoring (MRM). Reconstituted samples (5 uL) were injected into a 1.8 um BEH C18 column (50×2.1 mm; Waters) and separated using a linear gradient of 30-100% solvent B (100% acetonitrile, 0.1% formic acid, solvent A: 10% acetonitrile, 90% water, 0.1% formic acid) in 5 min followed by a 3 min hold at 100% B. The flow rate was 300 ul/min. The MRM transitions selected were m/z 335.3>135 (790), 337.3>160 (869), 397.2>220 (AEPU), 413.2>220 (hydroxy-adamantyl AEPU) and 346.3>169.4 (TPAU). Ionization parameters were optimized to a capillary voltage of 1 kV, cone voltage of 25 V, source temperature of 110° C., desolvation temperature of 300° C. and desolvation gas flow of 645 l/hr.

### Results

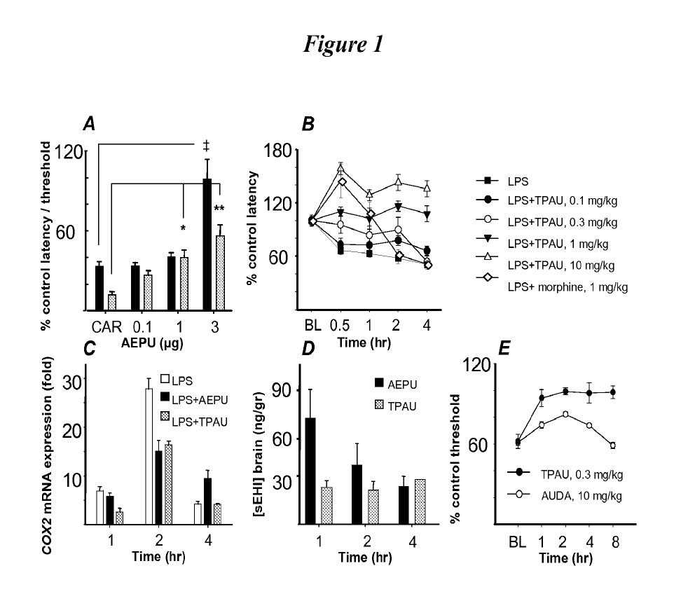

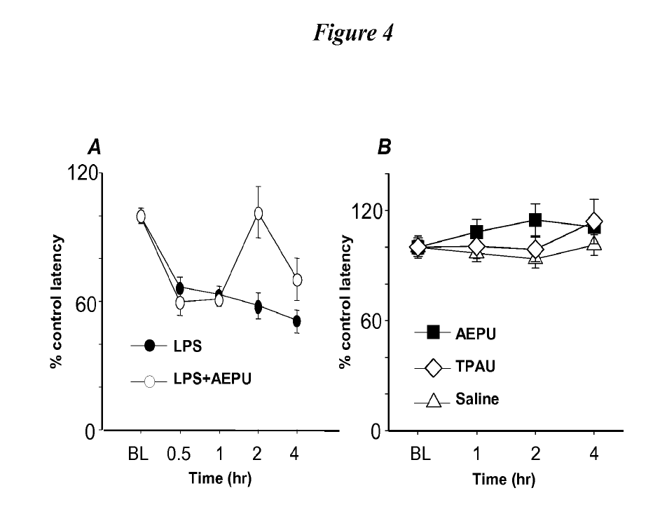

While monitoring epoxide/diol ratios of plasma fatty acids as markers of sEHI efficacy we surprisingly found an extensive reduction in pro-inflammatory fatty acid metabolites in severely inflamed mice treated with endotoxin (LPS) and sEHIs (Schmelzer K et al., Proc Natl Acad Sci USA 102:9772-9777 (2005)). These remarkable decreases, particularly in PGE₂ levels, compelled us to test if sEHI and/or EETs could reduce inflammatory pain. We found that sEHIs were highly potent antihyperalgesic agents in rodents by topical (Inceoglu B et al., Life Sci 79:2311-2319 (2006)), subcutaneous, or intrathecal administration. EETs alone and in combination with sEHI were also antihyperalgesic during inflammatory pain (Terashvili M et al., J Pharmacol Exp Ther 326:614-622 (2008)). The effect of the topically administered sEHI AEPU was demonstrated previously (Terashvili M et al., J Pharmacol Exp Ther 326:614-622 (2008); FIG. 4A). This inhibitor briefly increased noxious heat evoked paw withdrawal latencies in rats pre-treated with intraplantar (i.pl.) LPS. Although AEPU is metabolized rapidly, intrathecal administration of AEPU (0.1-3 μg) to rats through chronically implanted catheters resulted in a dose dependent decrease in carrageenan induced thermal hyperalgesia and mechanical allodynia (FIG. 1A). The metabolic lability of AEPU prompted us to design and synthesize a series of conformationally restricted sEHIs based on the acylpiperidine functionality (Jones P D et al., Bioorg Med Chem Lett 16:5212-5216 (2006)). These inhibitors are highly bioavailable and some have remarkably long half-lives (1 week). One of these sEHI, TPAU is highly effective in reducing inflammatory pain, in a dose dependent manner. Surprisingly, the activity of TPAU is comparable in analgesic potency to a moderate dose of morphine (1 mg/kg, subcutaneous) but with significantly longer efficacy (FIG. 1B). No loss of motor activity was observed after AEPU or TPAU administration to rats. Consistent with earlier findings the sEHI did not change nociceptive thresholds of rats in the absence of inflammatory pain (FIG. 4B). The polyethylene glycol structure of AEPU and the low melting point (low crystal stability) make it ideal for dermal formulations, while TPAU has excellent oral availability and pharmacokinetics (Table 1).

TABLE 1

| | | Melting | | | | |
|---|---|---|---|---|---|---|
| | | point | IC₅₀ (nM) | | | |
| Name | Structure | (° C.) | Human | Mouse | Rat | Mass |
| AUDA | 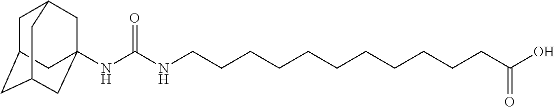 | 143 | 3 | 10 | 11 | 392.5 |
| AEPU | 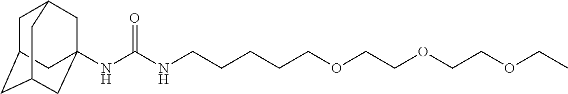 | 79 | 14 | 2.7 | 6.1 | 396.5 |
| TPAU | 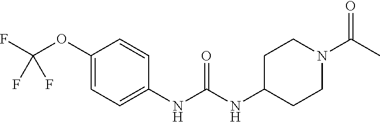 | 158 | 12 | 97 | 79 | 345.3 |

### Inhibitors of sEH Suppress the Induction of Spinal COX2 Message

In mice during sepsis or in rats during local inflammation, increased plasma PGE₂ levels were consistently reduced following sEHI treatment (Inceoglu B et al., Life Sci 79:2311-2319 (2006); Schmelzer K et al., Proc Natl Acad Sci USA 102:9772-9777 (2005)). However, peripheral inflammation and noxious stimuli are known to evoke a robust increase in the spinal cord COX2 gene expression and prostanoid production (Ramwell P W et al., Am. J. Physiol. 211:998-1004 (1966); Malmberg A B and Yaksh T L, Science 257:1276-1279 (1992); Samad T A et al., Nature 410:471-475 (2001)). Given the ability of sEHIs to reduce plasma levels of PGE₂ we hypothesized that sEHI would block spinal prostaglandin production. Relative spinal COX2 mRNA levels following LPS elicited pain and sEHI treatment were monitored as a measure of spinal prostanoid production. Similar to previous reports we observed a highly significant increase in spinal COX2 mRNA following intraplantar LPS administration (FIG. 1C), although this increase was different from that produced by complete Freund's adjuvant where the resulting slower induction is more prolonged but less efficacious (Samad T A et al., Nature 410:471-475 (2001)). Two structurally different sEHIs, AEPU and TPAU administered peripherally, markedly attenuated COX2 upregulation in the rat spinal cord (FIG. 1C). We found both sEHIs used efficiently penetrated into the brain, and thus these compounds are capable of direct action in the central nervous system (FIG. 1D). The suppression of spinal COX2 message is in parallel to an earlier report using another sEHI in which we showed a reduction in cox-2 protein level in liver of inflamed mice (Schmelzer K et al., Proc Natl Acad Sci USA 103:13646-13651 (2006)). The potent activity of intraspinal sEHI, the spinal repression of COX2 induction by sEHIs, along with detection of both sEHIs in the brain strongly supports a centrally mediated antihyperalgesic mechanism of action for sEHIs.

Inhibitors of sEH have COX2 Independent Antihyperalgesic Effects

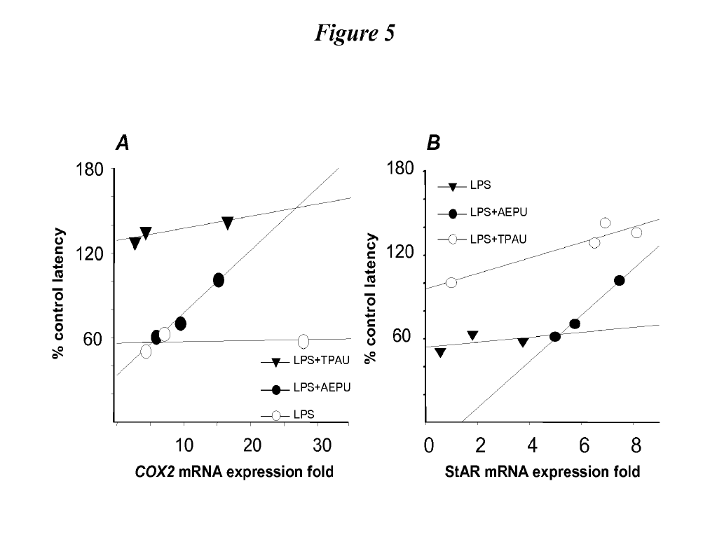

Given the lack of effect of sEHIs in the absence of facilitated pain states and the suppression of the COX2 induction in the spinal cord during inflammation, the inhibitors seemed to target transcriptional regulation of the COX2 gene. To test this hypothesis we asked if COX2 message levels correlated with pain behavior. Neither the two sEH inhibitors nor LPS treatment displayed a direct correspondence between spinal COX2 expression and antihyperalgesia (FIG. 5A). It is not unusual in the case of LPS to observe a weak linear relationship between spinal COX2 and pain scores because inflammation evokes a cascade of reactions including the release of numerous pronociceptive mediators with overlapping yet distinct temporal and spatial occurrence. However sEHIs were antihyperalgesic while COX2 message was induced, displaying a counter intuitive correspondence between increasing spinal COX2 and antihyperalgesia in these animals. While glucocorticoids are well-known repressors of COX2 expression and display a linear relationship between decreased pain related behavior and suppressed COX2 message (Hay C H and de Belleroche J S, Neuropharmacology 37:739-744 (1998)), sEHIs apparently lack this correlation (FIG. 5A). As a control we evaluated sEHIs using a neuropathic pain model, streptozocin induced diabetic neuropathy (Aley K O and Levine J D, J Pain 2:146-150 (2001)), that does not involve extensive COX2 upregulation. Surprisingly, we observed a significant decrease in mechanical allodynia of diabetic rats using the two structurally different sEHIs (FIG. 1E).

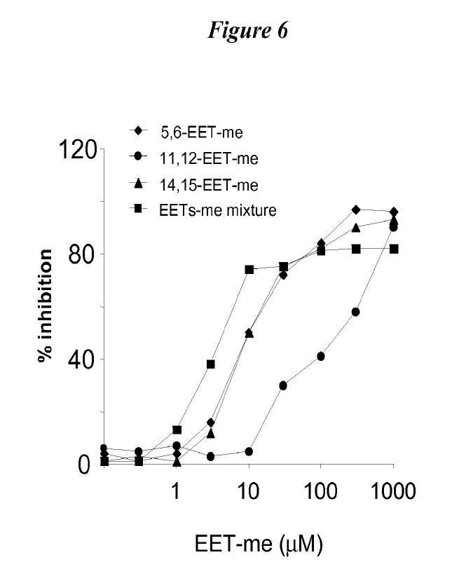

These results led us to look for an alternative mechanism of action. We hypothesized that EETs are the major mediators of the antihyperalgesic activity and screened the binding of EETs to a small set of cellular receptors. Given that EETs are highly hydrophobic and significantly similar in structure to ubiquitous fatty acids we did not anticipate that they would only have affinity to three of 48 targets tested (Inceoglu B et al., Prostag Other Lipid Mediat 82:42-49 (2007)). Of these potential targets we focused on translocator protein (TSPO), formerly known as the peripheral benzodiazepine receptor (Papadopoulos V et al., Trends Pharmacol Sci 27:402-409 (2006)). The mixture of synthetic EETs or their methyl ester analogs (EET-me), displaced a high affinity radioligand, [³H] PK 11195, from the TSPO with an IC₅₀ of 4.6 μM without affecting [³H]-flunitrazepam binding (FIG. 6). The TSPO is proposed to translocate cholesterol from the outer to the inner mitochondrial membrane for downstream synthesis of all steroids in the peripheral tissues but in the central nervous system (CNS) the endproducts are primarily neurosteroids (Papadopoulos V et al., Steroids 62:21-28 (1997); Papadopoulos V et al., Neuroscience 138:749-756 (2006); Papadopoulos V et al., Mol Cell Endocrinol, 265-266:59-64 (2007)). Earlier, TSPO ligands were shown to have antinociceptive and anti-inflammatory effects (Bressana E et al., Life Sci 72:2591-2601 (2003); da Silva M B et al., Mediat Inflamm, 13:93-103 (2004)).

Steroid Synthesis is Required for sEHI Mediated Analgesia

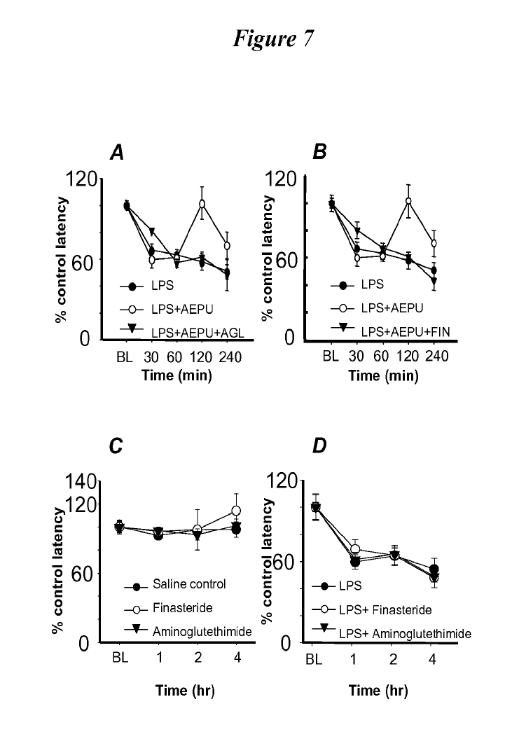

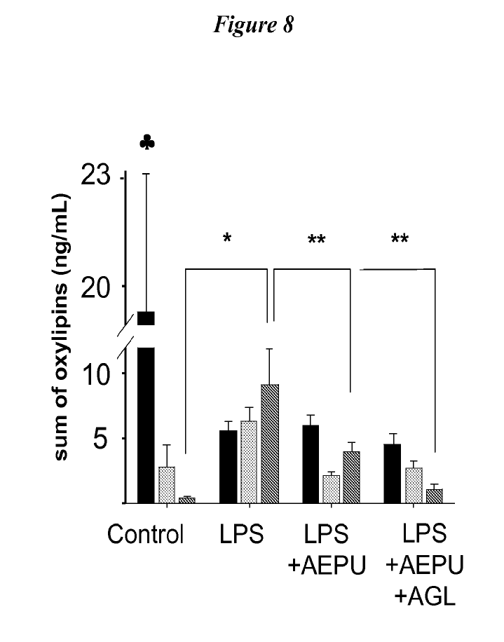

The dose-dependent displacement of [³H] PK 11195 from its binding site by EETs, while demonstrating a probable interaction of EETs and TSPO or a component of the steroidogenic machinery, did not reveal if EETs are agonistic or antagonistic in regard to the activity of this receptor. Additionally the observed effective concentration values (IC₅₀ of EETs mixture=4.6 μM) were far higher than what would be considered a tight receptor-ligand interaction. However, this assay is not an EET binding assay; rather it measures displacement of a high affinity ligand. In addition, EETs were shown to stimulate cortisol production in bovine adrenal fasciculata cells and estradiol and progesterone production in cultures of human luteinized granulose cells at similar concentrations (Van Voorhis B J et al., J Clin Endocrinol Metab 76:1555-1559 (1993); Nishimura M et al., Prostaglandins 38:413-430 (1989); Zosmer A et al., J Steroid Biochem Mol Biol 81:369-376 (2002)). Accordingly, we surmised EETs activate TSPO and that the effects of synthetic sEHIs and natural EETs were mediated partially through an increase in the production of analgesic neurosteroids in the central nervous system (CNS). We postulated that inhibition of acute steroidogenesis would partially antagonize sEHIs and tested this hypothesis using two steroid synthesis inhibitors that penetrate into the CNS (Finn D A et al., CNS Drug Rev 12:53-76 (2006); Unger C et al., Invest New Drugs 4:237-240 (1986)). As predicted, the antihyperalgesic activity of AEPU was abolished when aminoglutethimide (AGL, 10 mg/kg), a general steroidogenesis inhibitor or finasteride (FIN, 20 mg/kg), a 5α-reductase inhibitor, were co-administered (FIGS. 7A and B). These antagonists had no significant effect on the development of LPS induced thermal hyperalgesia nor did they change the responses of vehicle treated animals (FIGS. 7C and D). Aminoglutethimide, a selective inhibitor of cytochrome P450scc (side chain cleavage of cholesterol) did not change the plasma EET/DHET ratio in LPS and AEPU-treated rats indicating that antagonism by this compound could not be attributed to reduced EET production. This observation is in contrast to AEPU treatment which decreased plasma PGE₂ and DHET levels (FIG. 8). Furthermore aminoglutethimide did not antagonize the ability of the sEHI to reduce PGE₂ reiterating the presence of multiple mechanisms for the antihyperalgesic effects of inhibiting sEH (FIG. 8).

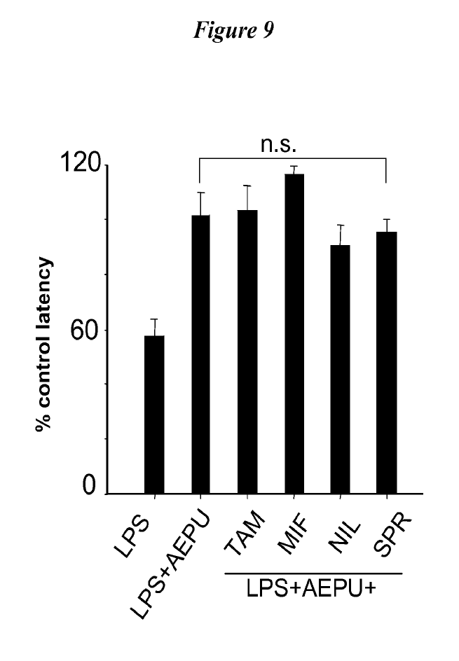

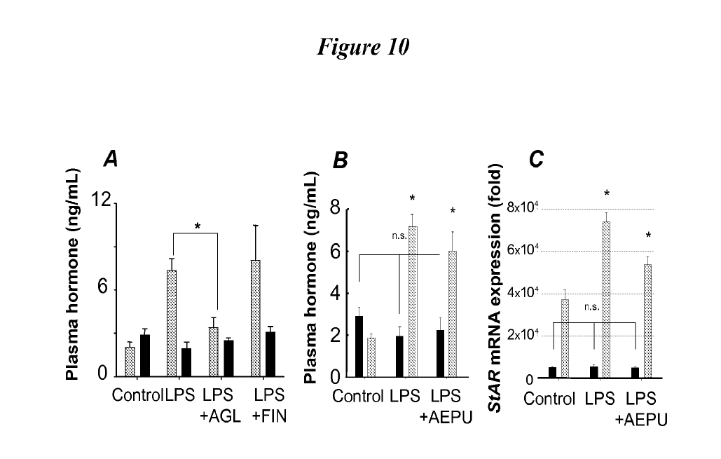

Next, we took a two-pronged approach to test if sEHI activity required the activation of nuclear steroid hormone receptors or if sEHIs influenced circulating steroid levels. None of the tested steroid receptor antagonists (10 mg/kg) significantly reversed the sEHI mediated antihyperalgesia (FIG. 9). Interestingly, peripheral inflammation increased circulating progesterone levels with no change in testosterone levels among treatments (FIG. 10A). Circulating hormone levels in animals treated with steroid synthesis inhibitors displayed the expected changes, but the hormones were not completely depleted during the course of the experiment (FIG. 10). Although AEPU treatment did not alter the levels of testosterone with or without LPS treatment it decreased plasma progesterone level (FIG. 10B). We also quantified a steroidogenesis marker gene, steroidogenic acute regulatory protein (StARD1) to confirm the plasma hormone assays. The mRNA levels of StARD1 in testis and adrenals were 5,000 and 37,000 fold higher than that of spinal cord which was used as the calibrator. Changes in expression level of StARD1 in two major peripheral steroidogenic tissues, the testis and adrenal glands, corresponded well with circulating progesterone and testosterone levels. There was no further enhancement of these levels by sEHI though the sEHI led to a minor decrease in adrenal StARD1 message level in parallel to the decrease observed in plasma progesterone level (FIG. 10C). These findings implicate a selective pattern of regulation of steroidogenesis by sEH inhibitors and/or EETs in addition to supporting the absence of an effect through classical steroid mediated gene expression or a general increase in steroidogenesis.

EETs and sEHIs Selectively Enhance Spinal StARD1 (Steroidogenic Acute Regulatory Protein) Expression

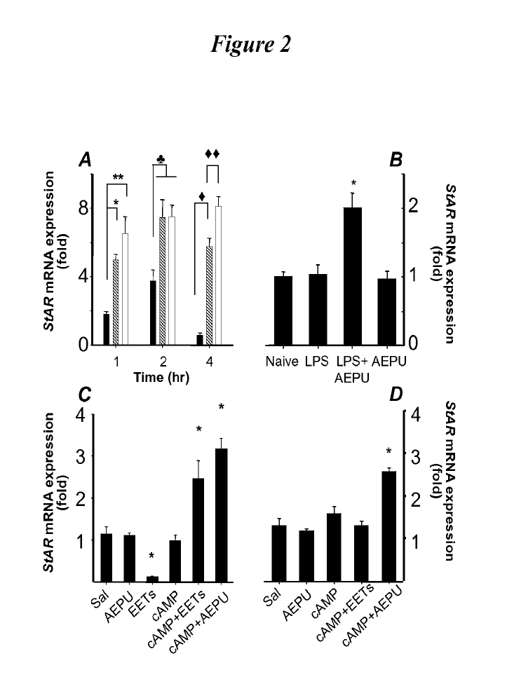

In contrast to above in vivo findings with sEH inhibitors, the in vitro stimulating effect of AA, its lipoxygenase and cytochrome P450 generated metabolites on steroidogenesis were recognized as early as the 1980s (Lin T Life Sci 36:1255-1264 (1985); Dix C J et al., Biochem J. 219:529-537 (1984)). At least part of this effect was traced to EETs, which stimulate cortisol production (Nishimura M et al., Prostaglandins 38:413-430 (1989)). Recently, EETs were shown to directly increase StARD1 gene expression and thus steroid synthesis in cell lines from reproductive tissues (Wang X et al., J Endocrinol 190:871-878 (2006)). It is proposed that acute steroidogenesis is largely dependent on rapid production and degradation of StARD1 message and protein and that TSPO and de novo StARD1 cooperatively facilitate the rate determining, finely tuned, on demand transport of cholesterol into the mitochondria (Clark B J et al., Endocrinology 138:4893-4901 (1997); Epstein L F and Orme-Johnson N R, J Biol Chem 266:19739-19745 (1991); Miller W L, Biochim Biophys Acta Mol Cell Biol Lipids 1771:663-676 (2007)). In the CNS however, the parallel steroid synthesis cascade produces a group of endogenous molecules termed neurosteroids which potentiate inhibitory GABA currents in neurons (Belelli D and Lambert J J, Nat Rev Neurosci 6:565-575 (2005)). We therefore asked whether increasing the level of EETs in the CNS by inhibiting sEH would enhance the expression of StARD1 mRNA. Interestingly, spinal StARD1 expression was already increased, though briefly, during inflammation (FIG. 2A) in parallel to the increase in adrenal StAR message. The two chemically dissimilar sEHIs greatly enhanced the increase in spinal StARD1 message in inflamed animals but not in non-inflamed controls that received AEPU alone. The increase in StARD1 message was positively correlated with the temporal occurrence of antihyperalgesia following administration of AEPU and TPAU (FIG. 5B). Notably, TPAU, the stronger repressor of COX2 message (FIG. 1C) displayed a shallower slope possibly because of a ceiling effect or superior down regulation of COX2. In brain, baseline StARD1 message levels were identical to those quantified from the spinal cord. Neither local inflammation nor AEPU alone elicited an increase in StARD1 message in the brain, although a two-fold increase was evident in inflamed animals treated with AEPU (FIG. 2B). Given the calculated half-life of StAR protein is ˜5 min. and that each StAR molecule is estimated to turn over ˜400 cholesterol molecules per minute in adrenal cells, we expect that the brief and minor expression changes mediated by sEHIs that are detected here can significantly amplify neurosteroid synthesis in the CNS thus lead to antihyperalgesia (Epstein L F and Orme-Johnson N R, J Biol Chem 266:19739-19745 (1991); Artemenko I P et al., J Biol Chem 276:46583-46596 (2001)).

EETs and sEHIs Redirect Elevated cAMP to an Analgesic Pathway

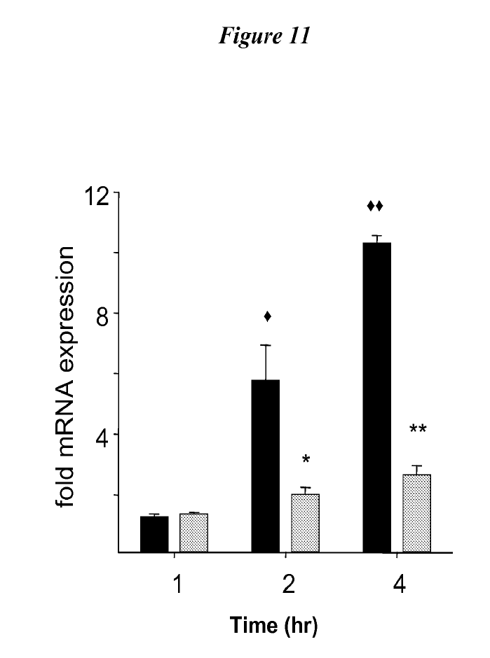

An important requirement for the interaction between EETs, TSPO activity and StARD1 expression may be the presence of elevated cAMP because expression and phosphorylation of StARD1 is greatly enhanced upon gonadotropic hormone stimulation, which increases intracellular cAMP levels (Stocco D M et al. Mol Endocrinol 19:2647-2659 (2005); Manna P R et al., J Mol Endocrinol 37:81-95 (2006)). Separately, the maintenance of hyperalgesia in inflammatory and neuropathic pain states is known to be largely regulated by the activation of the cAMP signaling pathway (Taiwo Y O et al., Neuroscience 32:577-580 (1989); Hucho T and Levine J D, Neuron 55:365-376 (2007); Song X-J et al., J Neurophysiol 95:479-492 (2006)). In the brain, intracellular cAMP level is known to rise rapidly in response to inflammation mainly because the cox-2 product PGE₂, activates E-Prostanoid receptors and initiates a cascade of events beginning with stimulation of adenylate cylase (Wellmann W and Schwabe U, Brain Res 59:371-378 (1973)). The resulting inflammatory pain can be blocked by an inactive cAMP analogue which prevents PKA activation (Taiwo Y O and Levine J D, Neuroscience 44:131-135 (1991)). Here we confirmed that peripheral inflammation led to an increase in spinal cord levels of intracellular cAMP by quantifying two cAMP responsive genes both of which were significantly induced during the course of inflammation (FIG. 11).

The prevailing outcome of elevated intracellular cAMP appears to be a sustained pain state. However, we hypothesized that increasing the level of endogenous EETs in the CNS in the presence of elevated cAMP may favor neurosteroid production by upregulating StARD1 expression. This should reduce nociceptive activity. Inferring that the concurrent presence of cAMP and EETs may be required for neurosteroid based antihyperalgesia we tested if StARD1 expression in the brain or the spinal cord of non-inflamed animals could be increased by direct spinal administration of 8-Br cAMP, EETs and sEHI. Because these animals were not inflamed and were under anesthesia, we predicted that changes in StAR expression would stem from injected cAMP and EETs/sEHI. Nociceptive thresholds of these animals were not determined because this assay was done under isoflurane anesthesia. As predicted, in non-inflamed rats 30 minutes after compound administration only co-administration of 8-Br cAMP (100 μg)+EETs (5 μg) and 8-Br cAMP+AEPU (1 μg) significantly increased spinal StARD1 levels (FIG. 2C). The EETs alone suppressed basal StARD1 expression while AEPU alone or cAMP alone were without effect. In the brain of the same animals again only the group that received cAMP and AEPU displayed an increase in StARD1 expression. Because AEPU in vivo is many fold more stable than EETs, this sEHI elicited a parallel increase in brain StARD1 whereas intraspinal EETs alone had no affect on brain StARD1 mRNA (FIG. 2D). Neither brain nor spinal StARD1 expression changed in response to saline or 8-Br cAMP administration. This observation is in contrast to cultured adrenal or testis cells where cAMP analogues are able to induce StARD1 expression and steroidogenesis. It is plausible that regulation of StARD1 in the CNS differs from that in reproductive and endocrine tissues. Overall these observations may explain the lack of efficacy of sEHIs in the absence of inflammation or neuropathy when intracellular cAMP levels are inadequate to drive neurosteroid production. Equally, during inflammation when EETs are not elevated or stabilized the influence of such an endogenous neurosteroid based antihyperalgesic mechanism may be marginal because EET levels in this case could become rate limiting. Interestingly, the expression levels of sEH message in spinal cord or brain were identical throughout the treatments in this study (data not shown) but inflammation caused a clear decrease in plasma oxylipins implying that spinal EETs may also be decreased during peripheral inflammation. As shown earlier, sEHI restored the plasma EET/DHET ratio (FIG. 8) (Schmelzer K et al., Proc Natl Acad Sci USA 103:13646-13651 (2006)). The hypothesis that AA release is required for sEHI mediated antihyperalgesia remains to be tested.

### Discussion

Although our original objective was not to delineate an endogenous neurosteroid-based antihyperalgesic pathway, two lines of evidence suggest that one exists and that it is modulated partly by EETs. Peripheral inflammation in our model caused a substantial and parallel increase in StARD1 expression in both the spinal cord and the adrenal gland (FIG. 2A and FIG. 10). Given that the adrenal StARD1 increase is accompanied by a surge in circulating progesterone levels and that StARD1 mRNA is a reliable marker for steroid production, we propose that a parallel increase in progesterone, an analgesic molecule and a precursor for neurosteroid production, may occur in the spinal cord. In fact we propose inhibition of sEH reveals the activity of a physiological system that is already in place to cope with inflammatory pain. Secondly, Poisbeau et al. reported that during peripheral inflammatory pain GABA_{A} receptor mediated synaptic inhibition was enhanced in lamina II dorsal horn neurons in a manner that can be reversed with finasteride, a neurosteroid synthesis inhibitor (Poisbeau P et al., J Neurosci 25:11768-11776 (2005)). The inhibitory influence of GABAergic tone on ascending pain transmission and the excitability of dorsal horn neurons are well established (Millan M J, Prog Neurobiol 66:355-474 (2002)). Given that neurosteroids are GABA agonists if levels of these molecules are elevated by inhibition of sEH this may enhance spinal GABAergic transmission in general and perhaps influence descending inhibition as well.

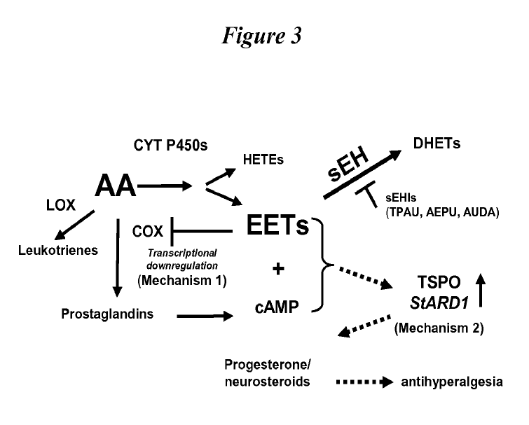

The tightly regulated nature of a likely TSPO/StARD1 based pathway is evident from the observations that the presence of elevated EETs and cAMP are both required to achieve StARD1 upregulation. Although the absence of linear correlation between spinal COX2 gene expression and pain scores strongly suggest that sEHIs act through an additional mechanism a correlation between StARD1 expression and pain scores does not necessitate a causal relationship. However, the binding of EETs to TSPO and antagonism of sEHIs by elimination of acute steroidogenesis strongly suggest so. Taken together, the hallmark of sEHI mediated antihyperalgesia could be that sEHIs afford the sustenance of a higher level of TSPO activation and/or StARD1 expression upon stabilizing natural EETs in the presence of elevated intracellular cAMP (FIG. 3) and enhance the production of unidentified factors, presumably including progesterone and other neurosteroids in the CNS, which are potent analgesics (Belelli D and Lambert J J, Nat Rev Neurosci 6:565-575 (2005)). Because an increase in intracellular cAMP levels in both inflammatory and neuropathic pain states is correlated with the occurrence of pain we predict inhibition of sEH may broadly result in antihyperalgesia in distinct pain models.

At least two endogenous mechanisms of pain control have so far been identified. These are the opioid and the endocannabinoid systems both of which are activated by stress, though they may also be active in various disease states (Hohmann A G et al., Nature 435:1108-1112 (2005); Lewis J W et al., Science 208:623-625 (1980)). Augmented neurosteroid production in the CNS during inflammation is likely another endogenous analgesic mechanism that exclusively operates during hyperalgesic states offering unique opportunities for therapeutical control of pain.

### Example 2

### Concurrent Inhibition of Soluble Epoxide Hydrolase and Phosphodiesterases Reveals Analgesic Properties of EETs in the Presence of Elevated Levels of cAMP

The cytochrome P450 generated metabolites of arachidonic acid, epoxyeicosatrienoic acids (EETs), are potent natural anti-inflammatory and analgesic molecules which posses multiple in vivo biological activities including suppression of induced COX2 message and protein upregulation as well as endorphin release. Although these bioactive lipids have very short half lives, preventing their degradation by inhibition of soluble epoxide hydrolase (sEH) stabilizes the EETs, and leads to antihyperalgesia in models of inflammatory and neuropathic pain. While sEH inhibitors (sEHi) have no antinociceptive properties in the absence of persistent pain states, here we tested the hypothesis that a factor associated with persistent pain, elevated cAMP, is required for EETs/sEHi to produce analgesia. In rats, concurrent administration phosphodiesterase inhibitors (PDEi), which increase intracellular cAMP levels, with sEHi lead to analgesia in a dose dependent manner. This activity was characterized pharmacologically. Notably, picrotoxin, a GABA_{A} antagonist, blocked the analgesic activity of sEHi+PDEi. We hypothesized that the observed increases in acute nociceptive thresholds could be mediated by a selective enhancement of spinal and supraspinal expression of a neurosteroid producing gene, StARD1 (steroidogenic acute regulatory protein). In rats receiving sEHi+PDEi treatment, the expression of the StARD1 gene in the spinal cord and brain was monitored along with the levels of a major neurosteroid, allopregnanolone. The expression of spinal and supraspinal StARD1 was increased in a dose dependent manner. Though this increase corresponded to analgesic activity, levels of spinal allopregnanolone surprisingly decreased in response to the sEHi+PDEi treatment. Overall, concurrent elevation of levels of EETs and cAMP by their respective inhibitors revealed a novel and unique interaction that seems to be related to the enhancement of GABA related activity. This combination thus could potentially be exploited as a general therapeutic strategy for the control of diverse types of pain states.

### Experimental Procedures

### Animals

This study was approved by the UC Davis Animal Care and Use Committee. Male and female Sprague-Dawley rats weighing 200-300 g were obtained from Charles River Inc., and maintained in UC Davis animal housing facilities with ad libitum water and food on a 12 hr:12 hr light-dark cycle. Data were collected during the same time of day for all groups.

### Chemicals

The sEH inhibitors AUDA (12-(3-adamantan-1-yl-ureido)-dodecanoic acid) and TPAU (1-trifluoromethoxyphenyl-3-(1-acetylpiperidin-4-yl) urea) were synthesized as previously reported (Morisseau C et al., Biochemical Pharmacology 63:1599-1608 (2002); Jones P D et al., Bioorganic & Medicinal Chemistry Letters 16:5212-5216 (2006)). Rolipram was purchased from Biomol International (Plymouth Meeting, Pa.). All other chemicals were obtained from Sigma-Aldrich (St. Louis, Mo.).

### Treatments and Behavioral Nociceptive Tests

Behavioral nociceptive testing was conducted by assessing thermal hindpaw withdrawal latencies (TWL) using a commercial Hargreaves apparatus (IITC, Woodland Hills, Calif.), or by determining mechanical hind paw withdrawal thresholds (MWT) using a digital paw pressure Randall-Selitto instrument (IITC, Woodland Hills, Calif.). On the day of the experiment, rats were transferred to a quiet room, acclimated for 1 hour and their baseline responses were measured. In pilot experiments, the intensity of the thermal stimulus was set to produce a baseline TWL of 7-8 sec. Baseline mechanical hind paw withdrawal thresholds varied between 60-70 grams of force. Immediately following baseline determination all compounds were administered subcutaneously in the following vehicles; Inhibitors of sEH were formulated in trans free oleate and administered in a total volume of 3004 Rolipram, picrotoxin, flucanozole, miconazole and finasteride were all dissolved in a sterile saline solution containing 25% DMSO and administered in a volume of 50 μl. Caffeine was dissolved in saline and administered in a total volume of 300 μl. All agents were administered subcutaneously into the back. In groups treated with the sEHi, cytochrome P450 inhibitors, finasteride or picrotoxin animals were pretreated one hour before other compounds. In groups treated with the PDEi, immediately following PDEi administration animals were placed in acrylic chambers on a glass platform maintained at a temperature of 30±1° C. for TWL measurement. Three to five TWL measurements were taken at 1-2 min interstimulus intervals following treatments and these were averaged for each animal at each time point. For MWT measurement, 45 minutes following treatment, the probe of the Randall-Selitto paw pressure meter was applied to the dorsal surface of the hind paw. The instrument was set to the maximum holding (MH) mode and the readout that elicited a hind paw withdrawal was designated as the threshold. Three MWT measurements were taken at 1-2 min interstimulus intervals and these were averaged for each animal. Data are presented as percent change from each animal's baseline response. Open field activity was quantified using a Plexiglas chamber (40×40×30 cm, length×width×height) imprinted with a 10×10 cm grid. Animals were placed in the middle of the chamber and observed for two minutes. The number of crossings were recorded when both hind paws crossed into a neighboring cell.

### Tissue Collection, Extraction, Analysis

Animals were sacrificed one hour following treatments by decapitation under deep anesthesia using isoflurane. The brain was rapidly removed and frozen on dry ice. The spinal cord was then rapidly removed following a laminectomy of the regions between L1-L5. Dorsal roots were excluded. Adrenal glands and testis from the same animals were also removed, flash frozen and stored at −80° C. The blood and brain levels of TPAU were determined as explained previously (Inceoglu B et al., Proc Natl Acad Sci USA 105:18901-18906 (2008)). Briefly, animals were deeply anesthetized under isoflurane and cardiac blood was collected. Animals were then perfused with cold normal saline prior to decapitation. Brains were then rapidly removed and flash frozen and stored at −80° C. until extracted. A section (−50 mg) of the prefrontal cortex was excised, weighed resuspended in a solution containing the internal standard compound 869 (1-adamantan-1-yl-3-(5-butoxy-pentyl)-urea, 250 ng/ml) and extracted using ethyl acetate as described. This extract was subjected to LC-ESI-MS/MS analysis using a Quattro Premier triple-quadrupole mass spectrometer (Waters) operating in positive electrospray ionization mode with multiple reaction monitoring (MRM). The MRM transitions selected were m/z 337.3>160 for compound 869, and 346.3>169.4 for TPAU. Ionization parameters were same as described previously set to a capillary voltage of 1 kV, cone voltage of 25 V, source temperature of 110° C., desolvation temperature of 300° C. and desolvation gas flow of 645 l/hr.

### Quantitative Real Time RT-PCR

Gene expression analysis was done as described previously (Inceoglu B et al., Proc Natl Acad Sci USA 105:18901-18906 (2008)). Briefly, RNA from whole spinal cord, brain and adrenal gland samples were extracted using a purelink Micro to Midi total RNA purification kit (Invitrogen, CA). The RNA samples were quantified by spectrophotometry and converted to cDNA using a high capacity cDNA reverse transcription kit from Applied Biosystems (CA, USA). Taq-man probe for StARD1 (Rn00580695_ml) was used according to manufacturer's instructions to quantify relative gene expression (Applied Biosystems CA, USA). Experiments were performed in triplicate with glyceraldehyde 3-phosphate dehydrogenase gene serving as the endogenous control. Mean fold expression values from corresponding vehicle treated animal tissues were used as calibrators.

Radioimmunoassay of Neuroactive Steroid Allopregnanolone ((3α,5α)-3-hydroxypregnan-20-one):

Radioimmunoassays were conducted as previously described (Janis G, C. et al., Alcoholism: Clinical and Experimental Research 22:2055-2061 (1998)). Briefly, spinal cord and brain samples were weighed and suspended in 2.5 ml of 0.3N NaOH, homogenized with a sonic dismembrator, and extracted three times with 3 ml aliquots of 10% ethyl acetate in heptane (vol/vol). Extraction recovery was monitored by the addition of 2000 cpm of [³H]allopregnanolone. The extracts were purified using solid phase silica columns (Burdick and Jackson, Muskegon, Mich.) and subsequently dried. Samples were reconstituted and assayed in duplicate by the addition of [³H]allopregnanolone and anti-allopregnanolone antibody (1:2000; Custom synthesis, Reproductive Endocrine Unit, Massachusetts General Hospital). Total binding was determined in the absence of unlabeled allopregnanolone and nonspecific binding was determined in the absence of antibody. The antibody binding reaction is allowed to equilibrate for 2 hours and cold dextran-coated charcoal was used to separate bound from unbound steroid. Bound radioactivity was determined by liquid scintillation spectroscopy. Steroid levels in the samples were extrapolated from a concurrently run standard curve and corrected for their respective extraction efficiencies. The sensitivity of the assay was 0.63 ng/ml. The intraassay coefficient of variation was <5%.

The radioimmunoassay of allopregnanolone employed a sheep polyclonal antibody that exhibits minimal cross reactivity with other circulating steroids (Janis G, C. et al., Alcoholism: Clinical and Experimental Research 22:2055-2061 (1998)), except the steroid (3a)₃-hydroxy-4-pregnen-20-one which binds to the antibody to a greater degree than allopregnanolone (169%). This compound may contribute to the measurement of allopregnanolone immunoreactivity, however since it's also a potent GABA_{A} receptor agonist, it would be expected to produce similar effects as allopregnanolone (Morrow A L et al., Mol Pharmacol 37:263-270 (1990)).

### Statistical Analyses

Data were analyzed by ANOVA followed by Tukey's post hoc test for between group comparisons using the SPSS analysis package (SPSS, Chicago, Ill.). Results are depicted as mean±SEM.

### Results

Elevated EETs and cAMP are Synergistically Antinociceptive in Rats

A non-inflammatory model was used to test the hypothesis that elevated intracellular cAMP and EETs will act synergistically in producing antinociception. Intracellular cAMP levels were increased by administering increasing doses of two CNS permeable PDEi; rolipram, a PDE-4 selective inhibitor and caffeine, a non selective PDEi. Inhibition of sEH was accomplished by administering two CNS permeable, structurally different sEHi, TPAU (10 mg/kg) and AUDA (40 mg/kg) each at a single dose.

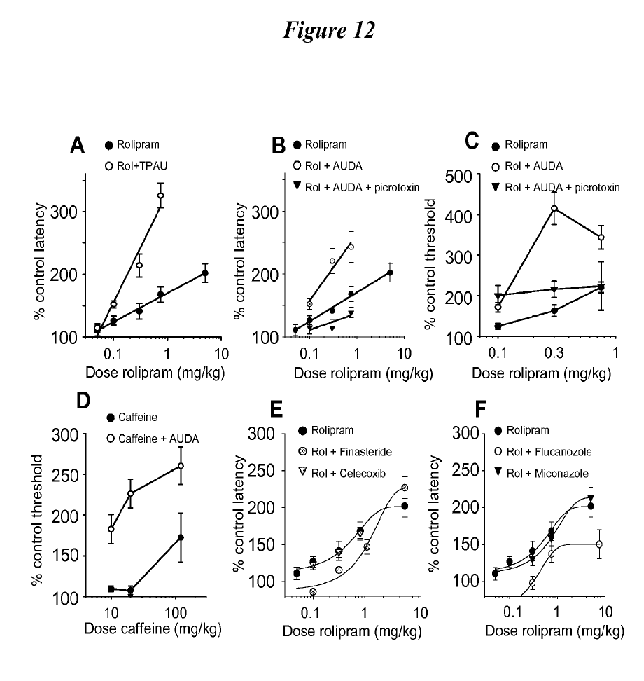

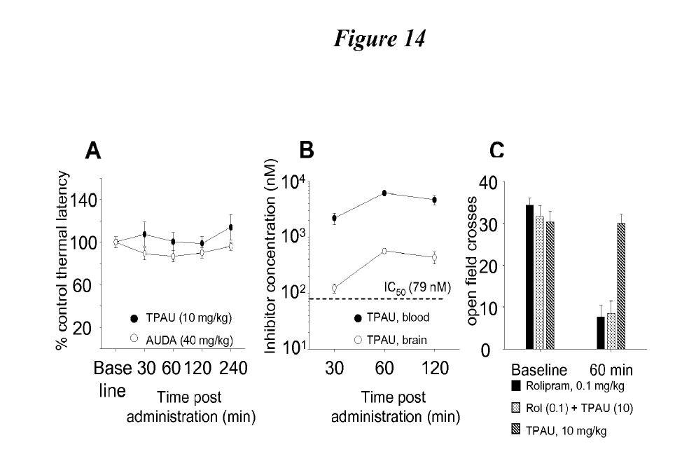

Consistent with the hypothesis that cAMP is a required factor for sEHi mediated antinociception, in the absence of a pain state, profound increases in both thermal and mechanical nociceptive thresholds of rats were evoked one hour following inhibitor administration when sEHi were combined with PDEi (FIG. 12). Although sEHi had no effect of their own on nociceptive thresholds (FIG. 14), both PDEi possessed a moderate degree of antinociceptive and a significant degree of motor depressant effects (FIG. 12) (Wachtel H, Psychopharmacology 77:309-316 (1982); Inceoglu B et al., Life Sciences 79:2311-2319 (2006)).

The PDEi rolipram dose dependently increased thermal withdrawal latency (FIG. 12A, ED₅₀=0.53 mg/kg). The TPAU+rolipram treatment also dose dependently increased thermal withdrawal latency (ED₅₀=0.34 mg/kg). The TPAU+rolipram combination was not only more potent than rolipram alone but also was 1.25 fold more efficacious (FIG. 12A). Another sEHi, AUDA, also displayed a parallel antinociceptive profile when combined with rolipram (ED₅₀=0.14 mg/kg) but this compound led to a less extensive increase in efficacy (FIG. 12B).

Rolipram also increased mechanical withdrawal thresholds of treated rats (ED₅₀=0.3 mg/kg, FIG. 12B). The AUDA+rolipram treatment enhanced this effect by about two fold (ED₅₀=0.14 mg/kg) and similarly was 1.35 fold more efficacious than rolipram alone (FIG. 12B). AUDA also enhanced the effects of caffeine at doses in which caffeine does not cause akinesia. At these two low doses of caffeine, drug induced hyperactivity prevented the determination of thermal withdrawal latencies. Therefore, only mechanical withdrawal thresholds are reported for caffeine groups. Rolipram, on the other hand, led to significant immobility, even at very low doses that was not altered by sEHi.

Two lines of evidence suggested the involvement of enhanced GABA-mediated transmission. Firstly, the antinociceptive effects of the sEHi+PDEi treatment was strongly blocked by picrotoxin (FIGS. 12B and C), a GABA antagonist at a dose that given alone did not change baseline responses. Picrotoxin depressed the maximal response of both the rolipram and AUDA+rolipram combination in the thermal withdrawal assay but only the AUDA+rolipram combination in the mechanical withdrawal assay suggesting a selective interaction of picrotoxin with the sEHi though more dose points are needed to precisely evaluate the nature of the interaction between picrotoxin and sEHi+PDEi treatment. Secondly, antagonism of rolipram's activity by finasteride, a neurosteroid synthesis inhibitor, in a competitive manner, suggests the involvement of steroids or neurosteroids (FIG. 12E). Earlier in an inflammatory pain model we demonstrated finasteride and the general steroid synthesis inhibitor aminoglutethimide both blocked the antihyperalgesic effect of an sEHi (Inceoglu B et al., Proc Natl Acad Sci USA 105:18901-18906 (2008)). On the other hand, Celecoxib, a selective cox-2 inhibitor, was without effect on rolipram's activity (FIG. 12E). This indicated a selective effect of sEHi in this system that is potentially independent of arachidonic acid release. Furthermore, rolipram's activity seemed, at least partially, to be mediated by endogenous epoxyeicosanoids because flucanozole, a CNS permeant EET synthesis inhibitor blocked the antinociception produced by rolipram in a non-competitive, non-surmountable manner (FIG. 12F). Conversely, miconazole another EET synthesis inhibitor that does not penetrate into the CNS failed to change rolipram's activity (FIG. 12F).

Elevated EETs and cAMP Activate CNS StARD1 Expression but not Neurosteroid Synthesis

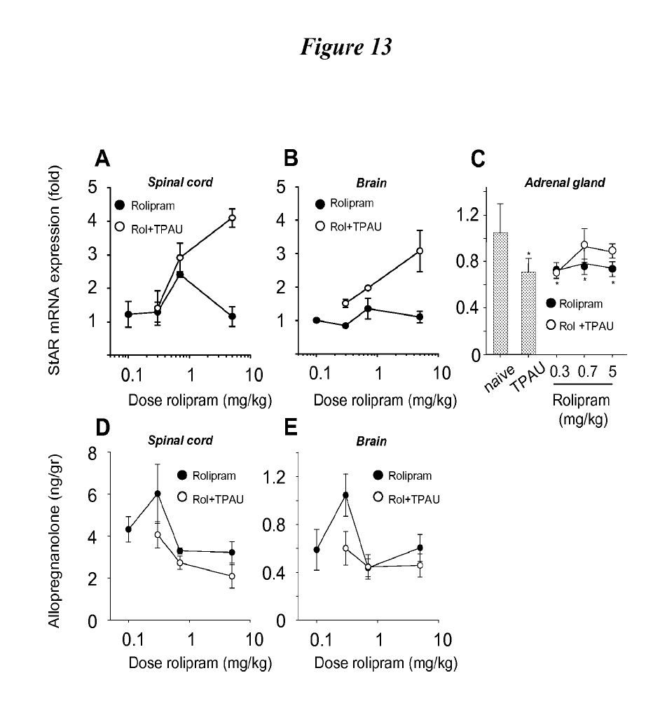

These findings encouraged us to investigate the expression of StARD1 message as a marker of steroidogenic activity. The mRNA level of StARD1 from the brain and the spinal cord was quantified one hour following administration of increasing doses of rolipram and a single dose of TPAU. Adrenal StARD1 from the same animals was also monitored as a marker of peripheral steroidogenesis. Rolipram significantly increased the expression of spinal StARD1 only at a single dose point and was ineffective in increasing brain StARD1 expression (FIG. 13). However, the TPAU+rolipram treatment led to small but significant and dose dependent increases in both spinal and brain StARD1 expression (FIGS. 13A and B). It should be stressed that these increases did not correspond well with the antinociceptive effects. However, the increases reported here are consistent with our prior findings demonstrating increased StARD1 expression when a cell permeable analogue of cAMP was administered to the spinal cord in the presence of EETs or an sEHi (Inceoglu B et al., Proc Natl Acad Sci USA 105:18901-18906 (2008)). By contrast, we found no evidence of adrenal increase in StARD1 expression, though a minor but significant decrease was observed in adrenal StARD1 expression with rolipram and TPAU or with the combined administration of the agents (FIG. 13C, P=0.03-0.05). These data from adrenal glands are consistent with our earlier finding in an inflammatory pain model that sEHi decreased the expression of adrenal StARD1 gene with a corresponding decrease in plasma progesterone (Inceoglu B et al., Proc Natl Acad Sci USA 105:18901-18906 (2008)).

To further test the hypothesis that sEHi in the presence of elevated cAMP levels leads to enhanced neurosteroid production, the spinal and brain levels of a prominent neurosteroid allopregnanolone were quantified (Selye H, Proc Soc Exp Biol Med 46:116-121 (1941); Paul S M and Purdy R H, FASEB J 6:2311-2322 (1992)). Allopregnanolone levels neither increased nor correlated with StARD1 expression levels in the spinal cord and the brain (FIGS. 13 D and E). The PDEi rolipram, in the spinal cord and the brain, did not lead to a dose dependent change in allopregnanolone levels. In animals that received sEHi+PDEi, spinal allopregnanolone levels decreased with increasing doses whereas in the brain no significant changes compared to vehicle or PDEi treated animals were observed. These findings indicate that StARD1 mRNA expression may not be a direct biomarker for neurosteroid production in the CNS.

### Discussion

The intracellular protein sEH rapidly degrades cytochrome P450 produced epoxygenated fatty acids (Spector A A and Norris A W, American Journal of Physiology-Cell Physiology 00402.02006 (2006)). Functional importance of sEH and epoxygenated fatty acids in various physiological processes including in the nervous system is progressively being recognized. Earlier, we demonstrated a surprising role for sEH and the arachidonic acid derived EETs in nociceptive signaling (Inceoglu B et al., Life Sciences 79:2311-2319 (2006); Schmelzer K R et al., Proc Natl Acad Sci USA September 12; ( ): 103:13646-13651 (2006); Inceoglu B et al., Prostaglandins & Other Lipid Mediators 82:42-49 (2007)). Few non-channel, non-neurotransmitter molecules are known to influence sensory function (Willis W D, Jr and Coggeshall, R. E., Sensory mechanisms of the spinal cord. New York: Kluwer Academic/Plenum Publishers (2004)). In inflammatory models of pain, inhibition of sEH not only suppressed the upregulation of COX2 gene expression in the spinal cord, but also unexpectedly upregulated an acute steroid producing gene, StARD1 (Inceoglu B et al., Proc Natl Acad Sci USA 105:18901-18906 (2008)), which we hypothesized would lead to the local production of neurosteroids, known to be positive allosteric modulators of GABA_{A} receptors (ref e.g. review: Murray et al., Pharmacology & Therapeutics 116:20-34 (2007)). The acute steroidogenic gene StARD1 requires elevated intracellular cAMP for expression and for the phosphorylation of its protein product (Arakane F et al., J Biol Chem 272:32656-32662 (1997)). The expression of StARD1 may also be activated by EETs (Wang X et al., The involvement of epoxygenase metabolites of arachidonic acid in cAMP-stimulated steroidogenesis and steroidogenic acute regulatory protein gene expression, 190:871-878 (2006)). These findings led us to test if increasing EET and cAMP levels concurrently would lead to first, an increased StARD1 expression in the nervous system, second, to increased neurosteroid production and last, to antinociception.

First, the hypothesis that sEHi require elevated levels of cAMP to increase nociceptive thresholds was tested. A non-inflammatory model was used in which the presumed limiting factors, intracellular cAMP and EETs, are concurrently elevated by simultaneously inhibiting their degradation. This model is advantageous in that animals were not inflamed thus no confounding anti-inflammatory effects of sEHi/EETs were present. As hypothesized, the sEHi+PDEi treatment led to highly significant increases in nociceptive thresholds over the baseline levels and over those produced by the PDEi alone (FIG. 12). In rats we used a single effective dose of the two sEHi. The doses used here (10 mg/kg TPAU and 40 mg/kg AUDA), we predict, were saturating doses (i.e., plasma inhibitor levels were 200 fold higher than rat sEH IC₅₀ values of AUDA and TPAU, unpublished results) during the course of the experiments. This allowed us to elevate cAMP levels in a stepwise fashion using the rapidly distributing, CNS permeable PDEi, rolipram (Krause W and Kiihne, G. Xenobiotica 18:561-571 (1988)). The observations on TPAU+rolipram were supported using a structurally different sEHi, AUDA, and a natural PDEi, caffeine though cAMP levels were not directly measured (FIG. 12D). Although the two sEHi used herein are structurally very different they both are powerful inhibitors of sEH (rat IC₅₀ TPAU=79 nM, rat IC₅₀ AUDA=11 nM). The activities reported herein were revealed by artificially stabilizing intracellular cAMP and EETs with the respective inhibitors of their degradation. However, in the course of inflammatory pain, a state where intracellular cAMP is physiologically elevated, stabilization of EETs by sEHi seems to be sufficient to yield antinociception (Zor U et al., Proc Natl Acad Sci USA 63:918-925 (1969); Inceoglu B et al., Proc Natl Acad Sci USA 105:18901-18906 (2008).

The antinociceptive activity of sEHi+PDEi treatment was then pharmacologically characterized. The antinociceptive effect of the sEHi+PDEi was found to be largely antagonized when GABA mediated transmission was blocked by picrotoxin. The dose of picrotoxin (0.25 mg/kg s.c.) used was not only ineffective on its own in changing nociceptive thresholds but was also possibly too low to cause analgesia through disinhibition of brainstem descending antinociceptive neurons (Koyama N et al., Pain 76:327-336 (1998)). This amount of picrotoxin was able to antagonize only the effects of sEHi+PDEi but not PDEi. Taken together our data suggest that picrotoxin attenuated analgesia primarily by blocking sEH inhibitor-mediated enhancement of GABA_{A} receptor function, and not by a general increase in neuronal excitability. Therefore our data suggest the involvement of GABA_{A} receptors in sEHi mediated antinociception. [also PDEi may lead to GABA receptor phosphorylation which is known to modulate GABAreceptor sensitivity to neurosteroids (see Petralia et al., Neuroendocrinology 84(6):405-14 (2006))]

In addition, preliminary experiments presented here on the antagonism of rolipram elicited antinociception by a CNS permeable EET synthesis inhibitor but not by a CNS impermeable EET synthesis inhibitor suggested at least part of the activity of the PDEis may be mediated by EETs (FIG. 12F). Interestingly, the effects of the sEHi+PDEi on the two nociceptive measures, the TWL and MWT were similar but not identical. Likewise the antagonism of antinociception by picrotoxin was different for TWL and MWT tests (FIG. 12B and C). Given that the Hargreaves' and the Randall-Selitto paw pressure tests are representative of, respectively, spinal and suprapinal-spinal information processing, it was interesting to observe a higher efficacy produced by the sEHi+PDEi in the MWT as opposed to the TWL. These data indicate that sEHi and PDEi lead to different but possibly overlapping effects.

The antinociceptive effects of both caffeine (at high doses) and rolipram have previously been recognized (Wachtel H, Psychopharmacology 77:309-316 (1982), Sawynok J and Yaksh T L, Pharmacol Rev 45:43-85 (1993); Sawynok J et al., Pain 61:203-213 (1995); Siuciak J A et al., Psychopharmacology 192:415-424 (2007)). However, a caution is that these effects are accompanied by significant motor depression, therefore could be considered non-selective. In this study, the sEHi enhanced the antinociceptive effects of rolipram without changing the profound motor depression produced by this PDEi (FIG. 14C). By contrast, caffeine at the two low doses led to hyperactivity. On the other hand, the sEHi synergized caffeine's antinociceptive effects even at these doses, in the absence of gross motor depression. At the high dose of caffeine the animals were immobile and the sEHi still enhanced nociceptive thresholds (FIG. 12D). These observations suggest that the measured effects may be selective and classified as antinociceptive rather than sedative.

Next, the expression of the acute steroid producing gene StARD1 in response to sEHi+PDEi was investigated. We found both supraspinal and spinal StARD1 expression was increased in response to sEHi+PDEi (FIG. 13). By contrast, no significant increase in StARD1 gene expression was detected in the adrenal glands, indicating a selective action of the sEHi in the nervous system (FIG. 13C). The sEHi TPAU here and another sEHi AEPU earlier both led to a small decrease in adrenal StARD1 expression (FIG. 13C) (Inceoglu B et al., Proc Natl Acad Sci USA 105:18901-18906 (2008)).

Although StARD1 is thought to lead to the production of all steroids, the location and the selectivity of the increase observed here strongly suggested that this increase could lead to more steroid/neurosteroid production in areas functionally relevant to nociception, the brain and the spinal cord. In this study the increase in spinal and supraspinal StARD1 expression corresponded well with increases in nociceptive thresholds produced by TPAU+rolipram (r²=0.97 for spinal StARD1 vs. TWL and r²=0.96 for brain StARD1 vs. TWL). Rolipram itself, despite being antinociceptive, did not increase brain StARD1 expression and biphasically changed spinal StARD1 expression (FIGS. 13A and B). However, the analgesic activity of the sEHi+PDEi could be detected at doses lower than those producing an increase in spinal or supraspinal StARD1 expression. It is possible that the sEHi+PDEi and the PDEi have different mechanisms of action in producing antinociception, thus produce different profiles of StARD1 expression in the nervous system.

More significantly, the direct analysis of levels of a prominent neurosteroid allopregnanolone in the brain and the spinal cord did not support the occurrence of a general increase in neurosteroid production in response to sEHi+PDEi or PDEi alone. Specifically, the sEHi+PDEi led to an unexpected decrease in allopregnanolone levels. It is also possible that the observed increases in spinal and brain StARD1 expression levels are coincidental, or are secondary to other changes in response to a general decrease in steroid levels upon sEHi treatment. These data may also indicate that increases in nervous system StARD1 do not necessarily result in neurosteroid production. Indeed, there is very little expression of StARD1 protein in brain and the mitochondrial benzodiazepine receptor may be responsible for initiating steroidogenesis in brain (Papadopoulos V L et al., Neuroscience 138:749-756 (2006)). It is also possible that during inflammatory pain other unknown factors may be required for increased synthesis of neurosteroids or steroidogenesis may be inhibited by inflammatory mediators. Finally, it is possible that sEHi+PDEi leads to regionally selective effects on neuroactive steroids that are not detected by global measurements of neurosteroids across brain or spinal cord. This idea would be consistent with the result that finasteride inhibits the antinociceptive effects that are observed.

It is widely recognized that cAMP signaling is highly compartmentalized, leading to diverse effects in a selective manner (Cooper D M F and Crossthwaite A J, Trends in Pharmacological Sciences 27:426-431 (2006)). Artificially increasing the levels of cAMP with various agents therefore lead to a multitude of biological effects though, in some cases contradictory results are reported. For example, although PDEi are being considered for therapeutic applications for their anti-inflammatory effects, in several animal models, cAMP analogues and PDEi lead to hyperalgesia by intrathecal or systemic administration (Taiwo Y O et al., Neuroscience 32:577-580 (1989); Taiwo Y O and Levine J D, Neuroscience 44:131-135 (1991); Song X-J et al., journal of physiology 95:479-492 (2006); Field S K, Expert Opinion on Investigational Drugs 17:811-818 (2008)). Similarly, intrathecal administration of the cell permeable cAMP analogue 8-Br-cAMP has been demonstrated to change nociceptive thresholds in a biphasic manner in sheep providing hypoalgesia at a single dose but either no effect or hyperalgesia at other doses (Dolan S and Nolan A M, Neuroscience Letters 309:157-160 (2001)). Remarkably rolipram, a selective PDE4 inhibitor, has been shown to be anti-inflammatory in a mouse model of LPS induced inflammation (Aoki M et al., J Pharmacol Exp Ther 298:1142-1149 (2001)). Furthermore rolipram has also been shown to increase nociceptive thresholds in rats in response to electric shock (Siuciak J A et al., Psychopharmacology 192:415-424 (2007)). The PDEis in this study were administered systemically, away from the hind paws, to the back of the animals to avoid inducing local hyperalgesia. Our results support the earlier findings that suggest systemic inhibition of phoshodiesterases may be antinociceptive.

Overall, profound antinociception in rats that can be reversed by picrotoxin provides functional evidence towards the hypothesis that inhibitors of sEH or natural EETs may enhance GABA function in the presence of increased levels of cAMP. The analgesic effects of sEHi+PDEi treatment were picrotoxin and finasteride reversible and consistent with the effects of neurosteroids on GABA channels. However, data on diminishing allopregnanolone levels decrease the plausibility of these activities being driven exclusively by allopregnanolone. The sEHi tested so far have provided efficacy in disease models only and they displayed no effect on baseline nociceptive responses (FIG. 14B). The remarkable increases in withdrawal latencies and thresholds reported herein support our earlier findings that EETs and sEH have important roles in nociceptive signaling though further studies are required to understand their mechanism of action(s). However these findings now bring up the possibility that systemically delivered sEHi+PDEi combinations may prove useful in cases such as post operative analgesia or during recovery from general anesthesia in some species, where somatosensory and motor depressant effects might be desirable.

### Example 3

### Elevated EETs and cAMP Enhance Pentobarbital Induced Loss of Righting Reflex and Delay Picrotoxin Induced Convulsions in Mice

### Methods

### Animals

This study was approved by the UC Davis Animal Care and Use Committee. Male and female Sprague-Dawley rats weighing 200-300 g were obtained from Charles River Inc., and maintained in UC Davis animal housing facilities with ad libitum water and food on a 12 hr:12 hr light-dark cycle. Data were collected during the same time of day for all groups.

### Chemicals

The sEH inhibitors 12-(3-adamantan-1-yl-ureido)-dodecanoic acid (AUDA) and 1-trifluoromethoxyphenyl-3-(1-acetylpiperidin-4-yl) urea (TPAU) were synthesized as previously reported (6,34). Rolipram was purchased from Biomol International (Plymouth Meeting, Pa.) and Nembutal was from Abbott Laboratories (Abbott Park, Ill.). All other chemicals were obtained from Sigma-Aldrich (St. Louis, Mo.). Rolipram, picrotoxin, flucanozole, miconazole and finasteride were all dissolved in a sterile saline solution containing 25% DMSO and administered subcutaneously in a volume of 50 μA.

### Loss of Righting Assay

Loss of righting was quantified as previously described, with a slightly lower dose of pentobarbital (Pinna, et al. (2004) Proc Natl Acad Sci USA, 101(16), 6222-6225).

### Sample Collection, Extraction, Analysis

Animals were sacrificed one hour following treatments by decapitation under deep anesthesia using isoflurane. Brain was rapidly removed and frozen on dry ice. Spinal cord was then rapidly removed following a full laminectomy of the regions between L1-L5. Dorsal roots were excluded. Adrenal glands and testis from the same animals were also removed, flash frozen and stored at −80° C.

### Results

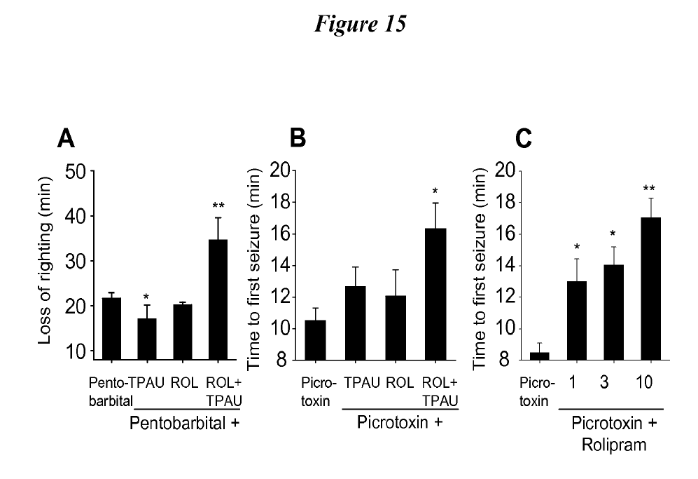

In rats, sEHi-PDEi combination led to significant increases in nociceptive thresholds. We asked if co-administration of sEHi-PDEi combination would also enhance GABA-related activity in mice. Loss of righting reflex in pentobarbital administered mice following sEHi and PDEi administration was quantified. Pentobarbital administration (40 mg/kg, intraperitoneal) expectedly resulted in the loss of righting reflex in C57BL/6 mice (FIG. 15A). Interestingly, a low dose of the sEHi, TPAU (3 mg/kg, s.c.) administered one hour prior to pentobarbital significantly reduced loss of righting while a high dose of the PDEi, rolipram (1 mg/kg, s.c.) had no effect. By contrast, consistent with observations in rats, the co-administration of the sEHi-PDEi (3 and 1 mg/kg, s.c.) resulted in a highly significant increase in loss of righting (FIG. 15A).

To confirm enhanced GABA related activity further experiments were conducted. Using a GABA antagonist, picrotoxin, we tested if picrotoxin induced epileptic seizures can be attenuated through co-administration of sEHi-PDEi. Picrotoxin (10 mg/kg, s.c.), expectedly, led to clonic seizures in C57BL/6 mice with a similar onset as reported previously for other mice (FIG. 15B). Unlike the loss of righting experiment, the sEHi, TPAU (3 mg/kg, s.c.) administered one hour prior to picrotoxin did not significantly change the time to onset of seizures. The high dose of the PDEi, rolipram (1 mg/kg, s.c.) also had no effect on time to onset of seizures. However, the co-administration of the sEHi-PDEi (3 and 1 mg/kg, s.c.) resulted in highly significant delay of seizure onset (FIG. 15B). Although, in sEH null mice picrotoxin induced seizure activity initiated earlier than it did in wild type conspecific mice, increasing doses of the PDEi in sEH null mice dose dependently delayed the onset of seizures (FIG. 15C). These results strongly suggest an enhancement in GABA channel related activity in animals that receive the sEHi-PDEi combination.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of reducing the severity and/or frequency of seizures in a subject in need thereof, said method comprising co-administering (a) (i) an inhibitor of sEH, (ii) an epoxygenated fatty acid, or (iii) both an inhibitor of sEH and an epoxygenated fatty acid, and (b) an agent that increases intracellular levels of cAMP.

2. The method of claim 1, wherein the epoxygenated fatty acid is an EET.

3. The method of claim 1, wherein the subject has epilepsy.

4. The method of claim 1, wherein said agent that increases intracellular levels of cAMP is an inhibitor of phosphodiesterase.

5. The method of claim 4, wherein said inhibitor of phosphodiesterase is a non-selective inhibitor of phosphodiesterase.

6. The method of claim 4, wherein said inhibitor of phosphodiesterase selectively inhibits a cAMP phosphodiesterase isozyme.

7. The method of claim 4, wherein said inhibitor of phosphodiesterase is an inhibitor of PDE4.

8. The method of claim 7, wherein said inhibitor of PDE4 is selected from the group consisting of rolipram, roflumilast, cilomilast, ariflo, HT0712, ibudilast, mesembrine, pentoxifylline, piclamilast, and combinations thereof.

9. The method of claim 4, wherein said inhibitor of phosphodiesterase is an inhibitor of PDE5.

10. A composition comprising (a) (i) an inhibitor of soluble epoxide hydrolase (“sEH”), (ii) an epoxygenated fatty acid, or (iii) both an inhibitor of sEH and an epoxygenated fatty acid, and (b) an agent that increases intracellular levels of cyclic adenosine monophosphate (“cAMP”).

11. The composition of claim 10, wherein said epoxygenated fatty acid is an epoxy-eicosatrienoic acid (“EET”).

12. The composition of claim 10, wherein said agent that increases intracellular levels of cAMP is an inhibitor of phosphodiesterase.

13. The composition of claim 12, wherein said inhibitor of phosphodiesterase is a non-selective inhibitor of phosphodiesterase.

14. The composition of claim 12, wherein said inhibitor of phosphodiesterase selectively inhibits a cAMP phosphodiesterase isozyme.

15. The composition of claim 12, wherein said inhibitor of phosphodiesterase is an inhibitor of PDE4.

16. The compositions of claim 15, wherein said inhibitor of PDE4 is selected from the group consisting of rolipram, roflumilast, cilomilast, ariflo, HT0712, ibudilast, mesembrine, pentoxifylline, piclamilast, and combinations thereof.

17. The composition of claim 12, wherein said inhibitor of phosphodiesterase is an inhibitor of PDE5.

18. A method of reducing depression, seizures in subjects with epilepsy, or of providing post-surgical analgesia during recovery from anesthesia, said method comprising co-administering (a) (i) an inhibitor of sEH, (ii) an epoxygenated fatty acid, or (iii) both an inhibitor of sEH and an epoxygenated fatty acid, and (b) an agent that increases intracellular levels of cAMP.

19. The method of claim 18, wherein the epoxygenated fatty acid is an EET.

20. The method of claim 18, wherein said agent that increases intracellular levels of cAMP is an inhibitor of phosphodiesterase.

21. The method of claim 20, wherein said inhibitor of phosphodiesterase is a non-selective inhibitor of phosphodiesterase.

22. The method of claim 20, wherein said inhibitor of phosphodiesterase selectively inhibits a cAMP phosphodiesterase isozyme.

23. The method of claim 20, wherein said inhibitor of phosphodiesterase is an inhibitor of PDE4.

24. The method of claim 23, wherein said inhibitor of PDE4 is selected from the group consisting of rolipram, roflumilast, cilomilast, ariflo, HT0712, ibudilast, mesembrine, pentoxifylline, piclamilast, and combinations thereof.

25. The method of claim 20, wherein said inhibitor of phosphodiesterase is an inhibitor of PDE5.

26. A method of enhancing the analgesic effects of EETs and inhibitors of sEH in a subject in need thereof, said method comprising co-administering (a) (i) an inhibitor of sEH, (ii) an epoxygenated fatty acid, or (iii) both an inhibitor of sEH and an epoxygenated fatty acid, and (b) an agent that increases intracellular levels of cAMP.

27. The method of claim 26, wherein the epoxygenated fatty acid is an EET.

28. The method of claim 26, wherein said agent that increases intracellular levels of cAMP is an inhibitor of phosphodiesterase.

29. The method of claim 26, wherein said inhibitor of phosphodiesterase is a non-selective inhibitor of phosphodiesterase.

30. The method of claim 26, wherein said inhibitor of phosphodiesterase selectively inhibits a cAMP phosphodiesterase isozyme.

31. The method of claim 26, wherein said inhibitor of phosphodiesterase is an inhibitor of PDE4.

32. The method of claim 31, wherein said inhibitor of PDE4 is selected from the group consisting of rolipram, roflumilast, cilomilast, ariflo, HT0712, ibudilast, mesembrine, pentoxifylline, piclamilast, and combinations thereof.

33. The method of claim 26, wherein said inhibitor of phosphodiesterase is an inhibitor of PDE5.

34. A method of enhancing anesthesia in a subject in need thereof, said method comprising co-administering (a) (i) an inhibitor of sEH, (ii) an epoxygenated fatty acid, or (iii) both an inhibitor of sEH and an epoxygenated fatty acid, and (b) an agent that increases intracellular levels of cAMP.

35. The method of claim 34, wherein the epoxygenated fatty acid is an EET.

36. The method of claim 34, wherein said agent that increases intracellular levels of cAMP is an inhibitor of phosphodiesterase.

37. The method of claim 34, wherein said inhibitor of phosphodiesterase is a non-selective inhibitor of phosphodiesterase.

38. The method of claim 34, wherein said inhibitor of phosphodiesterase selectively inhibits a cAMP phosphodiesterase isozyme.

39. The method of claim 34, wherein said inhibitor of phosphodiesterase is an inhibitor of PDE4.

40. The method of claim 39, wherein said inhibitor of PDE4 is selected from the group consisting of rolipram, roflumilast, cilomilast, ariflo, HT0712, ibudilast, mesembrine, pentoxifylline, piclamilast, and combinations thereof.

41. The method of claim 34, wherein said inhibitor of phosphodiesterase is an inhibitor of PDE5.

42. The method of claim 34, wherein the anesthesia is induced by a barbiturate.

43. The method of claim 34, further comprising administration of a barbiturate.
